(19) United States
(12) Patent Application Publication
(10) Pub. No.: US 2024/0277971 A1
(43) Pub. Date: Aug. 22, 2024

(54) ARCH FULCRUM CATHETERS

(71) Applicant: Daniel Ezra Walzman, Teaneck, NJ (US)

(72) Inventor: Daniel Ezra Walzman, Teaneck, NJ (US)

(21) Appl. No.: 18/565,149

(22) PCT Filed: May 27, 2022
(86) PCT No.: PCT/US2022/031231

§ 371 Date: Nov. 29, 2023

Related U.S. Application Data
(60) Provisional application No. 63/209,357, filed on Jun. 10, 2021.

(51) Int. Cl. A61M 25/01; A61F 2/01; A61F 2/95; A61M 25/00; A61M 25/10

(52) U.S. Cl. CPC A61M 25/0105; A61F 2/013; A61F 2/95; A61M 25/0023; A61M 25/1018; A61M 25/104

(57) ABSTRACT

A catheter configured to receive a supplemental medical device to facilitate treatment of a vascular anomaly within a blood vessel having a proximal hole and a distal hole. A lumen extends between the proximal hole and the distal hole and is configured to receive the supplemental medical device. At least one balloon element can be provided proximally of the distal end hole. At least one side hole is in communication with the lumen. Each side hole is configured to receive the supplemental medical device such that the supplemental medical device is extendable into the blood vessel through an elected side hole to increase access to a target site in the blood vessel and reduce rotational manipulation of the catheter required during an endovascular procedure. The catheter can be rotatable and steerable.

### CROSS-REFERENCE TO RELATED APPLICATIONS

This application claims the benefit of U.S. Provisional Application No. 63/209,357, filed on Jun. 10, 2021, and is a continuation-in-part of U.S. patent application Ser. No. 17/214,021, filed on Mar. 26, 2021, which claims the benefit of U.S. Provisional Application No. 63/109,387 filed on Nov. 4, 2020. The entire contents of each of these applications are incorporated herein by reference.

### FIELD OF THE INVENTION

This invention relates generally to endovascular devices and more particularly to a specifically shaped support catheter which obviates the need for an open surgical cutdown of the common carotid artery (CCA), while also employing a percutaneous technique and novel carotid access devices which use anatomical fulcrums and/or unique steering and/or rotational capabilities.

### BACKGROUND OF THE INVENTION

Minimally invasive treatments are increasingly popular including intravascular catheter treatments. Such treatments may be more effective than prior procedures, nonetheless, in some instruments they require an open surgical cutdown of the carotid artery which requires more anesthetic to perform typically than percutaneous procedures, with attendant anesthetic risks. Additionally, such procedures require surgical expertise, and presents additional risks of surgical injuries and/or infection at the cutdown site in the neck. The present disclosure in some embodiments relates to methods and systems for accessing the carotid arterial vasculature and establishing retrograde blood flow during performance of carotid artery stenting and other procedures.

Carotid artery disease commonly results in deposits of plaque which narrow the junction between the common carotid artery (CCA) and the internal carotid artery (ICA), an artery which provides blood flow to the brain. Such deposits may result in embolic particles being generated and entering the cerebral vasculature, leading to neurologic consequences such as transient ischemic attacks (TIA), ischemic stroke, or death.

Various therapies exist to ameliorate carotid artery disease related difficulties. The most common are carotid endarterectomy CEA and a carotid artery stenting CAS. Both expose patients to the risk of emboli being released into the cerebral vasculature via the internal carotid artery.

In response, several patents disclose a variety of devices and associated methods. For example, methods comprise trans-cervical access and blocking of blood flow through the common carotid artery while shifting blood from the internal carotid (see e.g., U.S. Ser. No. 12/835,660 (U.S. Pat. No. 8,784,355), Ser. No. 10/996,301 (U.S. Pat. No. 7,998,104), Ser. No. 12/366,287 (U.S. Pat. No. 9,669,191), and Ser. No. 15/044,493 (U.S. Pat. No. 9,655,755)).

The prior art discloses trans-carotid arterial revascularization. In particular, a small incision is made just above the collar bone and surgical dissection is used to surgically expose the common carotid artery. A soft, flexible tube (sheath) is placed directly into the carotid artery, and a clamp is applied to the external surface of the common carotid artery around the tube (sheath), and the tube (sheath) is connected to a system that will reverse the flow of blood away from the brain to protect against fragments of plaque that may come loose during the procedure. The blood is filtered and returned through a second tube (sheath) placed in the femoral vein in the patient's thigh or another vessel. Thereupon, the prior art also discloses balloon angioplasty and stenting performed while blood flow is reversed, and after the stent is placed successfully to stabilize the plaque in the carotid artery, the clamp is released and flow reversal is turned off and blood flow to the brain resumes in its normal direction. However, the prior art requires a surgical cut-down and dissection of the common carotid artery in the neck. Such surgery tends to disfigure the patient, requires additional anesthesia, additional training, and has a risk of damaging nerves.

Thus, there is a need for direct surgical access because of difficulties encountered with endovascular access, which can make adequate access difficult and high r risk in many cases.

There is a need for a less invasive percutaneous procedure, with lower risks of disfigurement, a lower risk of nerve injury, less use of anesthesia, and a simpler system requiring less training, thereby improving access to such treatments. The present invention addresses these unmet needs and provides advantages over the Walzman catheters described in the paragraph below.

The prior art discloses a set of Walzman arch fulcrum catheters, for example U.S. patent application Ser. Nos. 15/932,775 (Publication No. 2018/0243003) and Ser. No. 16/290,923, (Publication No. US 2019/0216499) which may be useful to overcome this difficulty. In particular, a version with a balloon on a distal end may be used. Using this device, a user may consistently obtain transfemoral carotid access with adequate support with little difficulties and lower risks, and achieve similar results via a percutaneous transfemoral access, with less needs for anesthetics and their attendant risks. Additionally, the prior art discloses a set of Walzman radial access catheters, which can make safe percutaneous access of either carotid artery feasible in the vast majority of patients, for example Ser. No. 16/501,592 (Publication No. 2019/0282266) and Ser. No. 16/501,577 (Publication No. 2019-0282265). Such catheters may also reduce access-site complications further. Such catheters can be further modified with at least one additional lumen substantially in the wall of the catheter, that can exit the wall of the catheter via at least one perforation in the outer wall of the catheter, to provide irrigation proximal to the balloon when said balloon is inflated, so as to minimize formation of clot proximal to said balloon. Such clots can form when a balloon occludes a vessel and causes stasis of blood.

### SUMMARY OF THE INVENTION

The present invention in some embodiments combines minimally invasive percutaneous endovascular carotid-artery access with rigorous blood flow-reversal, in order to protect the brain from embolic debris when introducing interventional devices into the carotid artery. In particular, the present invention uses reverse flow elements to prevent flow of blood to the brain, thus allowing maximal medical devices to be delivered to target areas more safely. Such reverse flow techniques include vaso-plugs, pumps, and irrigation distal to a lesion, among others.

The present invention may be embodied in the form of either of two preferred devices—one for right carotid stenosis and one for left carotid stenosis. The present invention includes a main (delivery') catheter, which may include one or more inflatable balloons mounted to an outer surface thereof, that is optimized for percutaneous access of the right and left carotid arteries in which a portion of the catheter is optimized to rest upon the lesser curvature of the aortic arch, in order to increase support for the delivery of additional medical devices (e.g., catheters, hypotubes, balloons, stents, etc.) through the catheter, while also preventing recoil and kickback and unwanted prolapse of the catheter and devices inserted therethrough into the aortic arch, thereby improving procedural efficacy and reducing procedural risks.

The current invention, in other embodiments, may use transfemoral percutaneous endovascular access via additional arch fulcrum access catheters referenced above, and additionally may use any of the radial access catheters described herein. Additional embodiments may alternatively use various catheters described in the prior art, along with novel devices and methods to increase flow reversal at the lesion site, while minimizing the necessary diameter of the delivery catheter, and while minimizing any potential sump effect from the brain.

In some embodiments of the current invention, a hollow wire is employed. The advantages of using a hollow wire include the ability to infuse fluids through it. This can
a. decrease sump effect during flow reversal to minimize distal tissue ischemia;
b. infuse blood if there is tissue ischemia;
c. infuse neuroprotective solutions, such as cooled fluids; and/or
d. infuse other material.

The benefits of using pressurized and/or pumped infusions is the ability to deliver higher rates (volume/time) of fluid through a small diameter lumen, thereby keeping the diameter of the devices as small as possible. This decreases risks at the treatment site and access site, and increases the range of access sites available (especially making treatment via radial artery access in most patients).

Additionally, using these methods and devices for carotid access can improve ease of percutaneous carotid access in many of the most difficult anatomical scenarios, thereby decreasing the risks of these percutaneous approaches. Catheters according to the present disclosure that are optimized for right carotid access via a transfemoral route will typically have a longer segment resting on the lesser arch of the aorta than corresponding catheter that are optimized for left carotid access. Embodiments include transfemoral and arm access arch fulcrum catheters.⋅The catheters may optionally have active steerability of their respective bends which in some embodiments can be achieved by the presence of wires in the wall of the catheter, with a mechanism to shorten the effective length of a wire to create a bend.

In one aspect of the present invention, a system for treating a vascular narrowing within a blood vessel is provided which includes a catheter and a supplemental medical device. The catheter includes: a proximal end hole; a distal end hole that is positioned opposite the proximal end hole; a circumferential balloon that is located proximally of the distal end hole; an operational lumen that extends through the catheter from the proximal end hole to the distal end hole; a first bend that curves in a first direction; and a second bend that curves in a second direction that is generally opposite to the first direction, wherein the second bend is positioned distally of the first bend and proximally of the circumferential balloon. The first bend and the second bend are configured to brace the catheter against an arch of the blood vessel to inhibit recoil of the catheter. The supplemental medical device is configured for insertion into the blood vessel through the operational lumen of the catheter.

In some embodiments, the supplemental medical device may be configured as a hypotube.

In some embodiments, the supplemental medical device may be configured as a catheter.

In some embodiments, the supplemental medical device may support a stent.

In some embodiments, the supplemental medical device may include at least one balloon element.

In some embodiments, the supplemental medical device may include a first balloon element and a second balloon element that is spaced axially from the first balloon element.

In some embodiments, the supplemental medical device may include a stent and at least one balloon element. In some embodiments, the at least one balloon element may include a first balloon element that is located distally of the stent and a second balloon element that is located distally of the first balloon element.

In some embodiments, the supplemental medical device may include a plurality of irrigation ports to facilitate fluid communication through the supplemental medical device into the blood vessel. In some embodiments, the plurality of irrigation ports may include a first plurality of irrigation ports that are located proximally of the first balloon element and a second plurality of irrigation ports that are located distally of the second balloon element.

In another aspect of the present disclosure, a system for treating a vascular narrowing within a blood vessel is provided which includes: a catheter; a first supplemental medical device that is configured for insertion into the blood vessel through the catheter; and a second supplemental medical device that is configured for insertion into the blood vessel through the first supplemental medical device. The catheter includes a tubular body having a first bend curving in a first direction and a second bend curving in a second direction generally opposite to the first direction. The first bend and the second bend are configured to brace the catheter against an arch of the blood vessel to inhibit recoil of the catheter.

In some embodiments, the medical device may further include a guide wire. In some embodiments, the catheter, the first supplemental medical device, and the second supplemental medical device may each be configured for insertion into the blood vessel over the guide wire.

In some embodiments, the first supplemental medical device may include a stent.

In some embodiments, the second supplemental medical device may include at least one balloon element. In some embodiments, the at least one balloon element may include a first balloon element and a second balloon element that is located distally of the first balloon element.

In some embodiments, the second supplemental medical device may be configured such that the first balloon element and the second balloon element are positionable distally of the stent.

In some embodiments, the second supplemental medical device may include a plurality of irrigation ports to facilitate fluid communication through the second supplemental medical device into the blood vessel. In some embodiments, the plurality of irrigation ports may include a first plurality of irrigation ports that are located proximally of the first balloon element and a second plurality of irrigation ports that are located distally of the second balloon element.

In another aspect of the disclosure, a system for treating a vascular narrowing within a blood vessel is provided. The system includes: a catheter; a first supplemental medical device that is configured for insertion into the blood vessel through the catheter; and a second supplemental medical device that is configured for insertion into the blood vessel through the first supplemental medical device, wherein the first supplemental medical device preferably includes a stent and the second supplemental medical device preferably includes at least one balloon element. The catheter includes a tubular body and a circumferential balloon that is secured to the tubular body. The tubular body includes a plurality of bends curving in a plurality of different directions such that the catheter is configured for bracing against an inner wall of the blood vessel to inhibit recoil of the catheter.

In some embodiments, the second supplemental medical device may include a first plurality of irrigation ports and a second plurality of irrigation ports that are located distally of the first plurality of irrigation ports. In some embodiments, the first plurality of irrigation ports and the second plurality of irrigation ports may be configured to facilitate fluid communication through the second supplemental medical device into the blood vessel. In some embodiments, the at least one balloon element may be positioned between the first plurality of irrigation ports and the second plurality of irrigation ports.

In another aspect of the disclosure, a catheter is disclosed that is configured to receive a supplemental medical device to facilitate the treatment of a vascular narrowing within a blood vessel during an endovascular procedure. The catheter includes: a proximal end hole; a distal end hole that is positioned opposite the proximal end hole; an operational lumen that extends between the proximal end hole and the distal end hole and which is configured to receive the supplemental medical device and a plurality of side holes that are in communication with the operational lumen and are arranged in a staggered pattern. Each side hole is configured to receive the supplemental medical device such that the supplemental medical device is extendable into the blood vessel through an elected side hole to increase access to a target site in the blood vessel and reduce rotational manipulation of the catheter required during the endovascular procedure. At least one balloon element can be provided that is located proximally of the distal end hole.

In some embodiments, the plurality of side holes may be staggered along a longitudinal axis of the catheter such that the plurality of side holes are spaced axially from each other.

In some embodiments, the plurality of side holes may be staggered along a circumference of the catheter such that the plurality of side holes are spaced circumferentially (radially) from each other.

In some embodiments, the plurality of side holes may be staggered such that they are circumferentially aligned arranged in at least one band.

In some embodiments, the plurality of side holes may be staggered such that they include a first plurality of side holes that are circumferentially aligned and arranged in a first band and a second plurality of side holes that are circumferentially aligned and arranged in a second band that is spaced axially from the first band along the longitudinal axis of the catheter.

In some embodiments, the plurality of side holes may be staggered along both the circumference of the catheter and the longitudinal axis of the catheter such that the plurality of side holes are spaced circumferentially (radially) and axially from each other. This can form a (generally) helical arrangement.

In some embodiments, the at least one balloon element may include a plurality of balloon elements. In some embodiments, the plurality of balloon elements may be staggered along a circumference of the catheter such that the plurality of balloon elements are spaced circumferentially (radially) from each other. In some embodiments, the plurality of balloon elements may be staggered such that they are circumferentially aligned and arranged in a band.

In some embodiments, the plurality of balloon elements may be staggered along a) both a longitudinal axis of the catheter such that the plurality of balloon elements are spaced circumferentially and b) axially from each other. This can form a (generally) helical arrangement.

The balloon element(s) can be on an outer catheter positioned over the catheter or on the catheter having the side hole(s).

In another aspect of the disclosure, a catheter is disclosed that is configured to receive a supplemental medical device to facilitate the treatment of a vascular narrowing within a blood vessel during an endovascular procedure. The catheter includes: a proximal end hole; a distal end hole that is positioned opposite the proximal end hole; an operational lumen that extends between the proximal end hole and the distal end hole and which is configured to receive the supplemental medical device; a plurality of balloon elements selectively inflatable to secure the catheter within the blood vessel; and at least one side hole that is configured to receive the supplemental medical device such that the supplemental medical device is extendable therethrough into the blood vessel.

In some embodiments, the plurality of balloon elements may be staggered along a circumference of the catheter such that the plurality of balloon elements are spaced circumferentially (radially) from each other.

In some embodiments, the plurality of balloon elements may be staggered such that they are circumferentially aligned and arranged in a band.

In some embodiments, the plurality of balloon elements may be staggered along a longitudinal axis of the catheter such that the plurality of balloon elements are spaced circumferentially (radially) and axially from each other. This can form a (generally) helical arrangement.

In some embodiments, the at least one side hole may include a plurality of side holes that are arranged in a staggered pattern along a longitudinal axis of the catheter and/or a circumference of the catheter.

The catheters disclosed herein can have a wire to apply a torsional force to rotate the catheter.

The catheters disclosed herein can have a wire to bend (deflect/steer) a distal segment of the catheter.

The catheters disclosed herein can provide support to the supplemental medical device to prevent kickback and prolapse.

In another aspect of the disclosure, a method of treating an aneurysm within a blood vessel is disclosed that includes: inserting a catheter into the blood vessel; inserting a supplemental medical device into an operational lumen of the catheter; electing a side hole from a plurality of side holes that are in communication with the operational lumen; and passing the supplemental medical device through the side hole elected from the plurality of side holes such that the supplemental medical device extends therethrough into the blood vessel.

In some embodiments, electing the side hole may include electing the side hole from a plurality of side holes that are staggered along a longitudinal axis of the catheter.

In some embodiments, electing the side hole may include electing the side hole from a plurality of side holes that are staggered along a circumference of the catheter.

In some embodiments, electing the side hole may include electing the side hole from a plurality of side holes that are arranged in a staggered pattern circumferentially (radially) and longitudinally in a (generally) helical pattern.

In some embodiments, the method includes inflating at least one balloon element that is supported on the catheter to secure the catheter within the blood vessel. In some embodiments, inflating the at least one balloon element may include inflating at least one of a plurality of balloon elements that are staggered along a circumference of the catheter and/or along a longitudinal axis of the catheter to deflect the catheter within the blood vessel.

In accordance with another aspect of the present invention, a method of treating a vascular anomaly within a blood vessel is provided comprising inserting a catheter into the blood vessel containing a vascular anomaly; electing a side hole from at least one side hole in communication with the lumen; passing a supplemental medical device through the side hole elected such that the supplemental medical device extends into the vascular anomaly therethrough; and rotating a segment of the catheter to rotate the side hole to a desired orientation.

In some embodiments, inserting the catheter into the blood vessel may include aligning the side hole elected with a vascular anomaly. In some embodiments, aligning the side hole elected with the vascular anomaly may include aligning the side hole elected with an aneurysm. In some embodiments, passing the supplemental medical device through the side hole may include inserting the supplemental medical device into the aneurysm through the side hole elected. In some embodiments, the method may further include inserting an embolic device into the aneurysm through the supplemental medical device to treat the aneurysm.

In some embodiments, the method may further include positioning or bracing the catheter against an inner wall of the blood vessel to inhibit recoil of the catheter and the supplemental medical device.

In another aspect of the disclosure, a catheter is disclosed that is configured for use during an endovascular procedure to treat a vascular abnormality in a blood vessel. The catheter includes an operational lumen that is configured to receive a supplemental medical device to facilitate treatment of the vascular abnormality and at least one side hole. The at least one side hole extends through the wall of the catheter and into communication with the operational lumen and is configured to receive the supplemental medical device such that the supplemental medical device is extendable into the blood vessel through the at least one side hole to treat the vascular abnormality.

In some embodiments, at least one balloon element is supported on an outer surface of the catheter and an inflation lumen extends through a wall of the catheter to selectively inflate the at least one balloon element

In some embodiments, the catheter may further include at least one pull or push wire that is configured to apply torsional force to the catheter and thereby rotate the catheter to vary a rotational position of the at least one side hole. The wire in some embodiments is embedded in the wall of the catheter.

In some embodiments, the at least one wire may include a first pull or push wire that is configured to rotate the catheter in a first direction and a second pull or push wire that is configured to rotate the catheter in a second direction generally opposite to the first direction. In other embodiments, the first and second pull or push wires rotate the catheter in the same (first) direction.

In some embodiments, the catheter may include at least one steering wire which can be pushed or pulled to deflect (bend) the distal end of the catheter. In some embodiments, the at least one steering wire is embedded in a wall of the catheter. Multiple steering wires can be provided to steer (bend) different segments of the catheter.

In some embodiments, the at least one balloon element may include a plurality of balloon elements that are staggered circumferentially and/or radially along a circumference of the catheter. In some embodiments, the at least one side hole may include a plurality of side holes that are staggered circumferentially and/or radially along a circumference of the catheter.

### BRIEF DESCRIPTION OF THE DRAWINGS

FIG. IA is a cross-sectional view of one embodiment of a catheter of the described invention that includes a tube (tubular body) I. The catheter is disposed in a blood vessel V (e.g., a bovine, Type III 7000 aortic arch) such that the second segment rests on the arch fulcrum 2000 with a third bend 30 and fourth segment 400 deployed in the left common carotid artery 5000 with a bovine origin. Also identified are descending aortic artery 1000, right subclavian artery 3000, right vertebral artery 3500, right carotid artery 4000, innominate (brachiocephalic) artery 6000, Type Ill arch 7000, left subclavian artery 8000, and left vertebral artery 8500.

FIG. 1B is a cross-sectional view through tube I taken along line 1B in FIG. IA according to an alternate embodiment of the disclosure.

FIG. 2 is a cross-sectional view of one embodiment of tube I of the present invention in place having the second segment 200 resting on the fulcrum of arch 2000 with an obtuse (inner) third bend 30 and fourth segment 400 deployed in the right subclavian artery 3000.

FIG. 3A is a cross-sectional view of one embodiment of tube I of the present invention in place having the second segment 200 resting on the fulcrum of arch 2000 with a second bend 20, and third segment 300 deployed in the right carotid artery 4000.

FIGS. 3B and 3C are enlarged views of the areas of detail identified in FIG. 3A.

FIG. 4 is a cross-sectional view of an embodiment of tube I of the preset invention having a distal element 400 comprising multiple bends and segments deployed in the right subclavian artery 3000.

FIG. 5 is a cross-sectional view of one embodiment of the tube I of the present invention having at least one lumen in the interior of tube 1, the tube I having a first segment 100 entering from an arm vessel or axillary artery or vein, a second segment 200 disposed between first bend IO and second bend 20, resting on the fulcrum of arch 2000, and third segment 300 deployed in the left common carotid artery 5000.

FIG. 6 is a cross-sectional view of one embodiment of tube I of the present invention having at least one lumen in the interior of tube I, the tube I having a first segment 100 entering from an arm vessel or axillary artery or vein through left subclavian artery 8000, a first side hole 170 proximal to the entry point of left vertebral artery 8500, a second segment 200 resting on aortic arch 2000, the segment including side hole 270, and a second bend 20 directing third segment 300 upward into innominate (brachiocephalic) artery 6000 wherein end hole 405 is disposed.

FIG. 7 is a cross-sectional view of the tube 1 of the present invention having two additional side holes 170 and 171 disposed within right subclavian artery 3000; balloon element 333 that is supported on the catheter (i.e., on the tube 1) proximal to end hole 405 disposed within the left subclavian artery 8000, and an inflation lumen (not shown) for balloon element 333 passing substantially through the wall of the intraluminal portion of tube 1 to balloon element 333. The balloon element(s) 333 are selectively inflatable to thereby secure the device within the blood vessel V (FIG. IA).

FIG. 8 illustrates the angle ranges for bend 10 (e.g., angle range 190 degrees to 280 degrees) and the angle ranges for bend 20 (e.g., angle range 70 degrees to 150 degrees), wherein the opposite angle range for bend 10 is 1111 and the opposite angle range for bend 20 is 2111. The arrow in FIG. 8 denotes direction of passage of devices from outside the body relative to the angles 1111 and 2111. It should be noted that bend numbers 10 and 20 have corresponding opposing angle ranges such as 1111 and 2111, respectively. This nomenclature distinction has been to insure clarity of disclosure.

FIG. 9 is a cross sectional view of one embodiment of tube 1 of the present invention having an arm access arch fulcrum support with three bends.

FIG. 10 is an illustration of an embodiment for transfemoral percutaneous treatment of the right carotid in most anatomies, with the balloon 333 of tube 1 deflated. The balloon 333 is disposed at (or adjacent to) the distal end hole 405. First internal curve 3111 of first bend 10 has a curvature of 70 to 120 degrees and second internal curve 4111 of second bend 20 has a curvature of 65 to 130 degrees. Bends 10 and 20 have corresponding, opposing angle ranges or internal curves such as 3111 and 4111, respectively.

FIG. 11 illustrates an embodiment for the left carotid artery with the balloon 333 of tube 1 inflated. In this embodiment, internal curve 5111 of bend 10 has a curvature of between 60 and 120 degrees.

FIG. 12 illustrates external elements of the present invention, including filter 9222, Y-connector 9223, external termination device of first catheter such as a Luer Lock element 9224, venous sheath 9225, flow regulator 9226, and stopcock 9227.

FIG. 13 illustrates an irrigation catheter 9300, having a distal end 9333, and a plurality of fluid-delivery ports 9334 (depicted in position in FIG. 18).

FIG. 14 illustrates an embodiment of the irrigation catheter 9300 of FIG. 13 further including angioplasty balloon element 9555, occlusion balloon element 9556, a plurality of irrigation ports 9334i, and plurality of irrigation ports 9334ii disposed proximal to tapered distal tip 9557.

FIG. 15 illustrates the interior of a narrowed internal carotid-artery lumen 7892, showing an inflated balloon 333 of tube 1 of the present invention disposed proximal to distal end hole 405 and narrowed area 7892, with blood flow direction depicted by the arrows. In particular, the narrowed lumen 7892 is restricted by cholesterolic plaque 7891 extending from carotid bulb 7890.

FIG. 16 illustrates the narrowed internal carotid-artery lumen 7892 of FIG. 15 with a delivery catheter 8970i of the present invention that supports and delivers a stent over delivery wire 8972, and further showing blood flow direction at the lesion by the arrow.

FIG. 17 illustrates the narrowed internal carotid-artery lumen 7892 of FIG. 15, with an inflated angioplasty occlusion balloon 8973 (e.g., a first balloon element) disposed upon fluid delivery catheter (hypotube) 8974, the blood flow direction shown by the arrows.

FIG. 18 illustrates the narrowed internal carotid-artery lumen 7892 of FIG. 15, further showing delivery wire 8972, guiding irrigation catheter 9300 which has multiple fluid-delivery ports 9334 (one shown in FIG. 13) disposed thereon both proximally to the temporary occlusion (second) balloon element 8975 and distally to the distally delivered deflated angioplasty (first) balloon element 8973, and balloon elements 8973 and 8975 mounted thereto. More specifically, (first) balloon element 8973 is located distally of, and is spaced axially from, (second) balloon element 8975 along the length of the irrigation catheter 9300. Stent 8971 is illustrated as being exposed (delivered, unsheathed) from hypotube 8970i, which may replace or supplement delivery catheter 8970. Hypotube 8970i and irrigation catheter 9300 each have a sufficient diameter to be able to be advanced over delivery wire 8972.

FIG. 19 is a perspective view of an alternate embodiment of the present invention in which the catheter (seen in FIG. IA) includes a plurality of (circumferentially staggered) side holes.

FIG. 20 is a perspective view of an alternate embodiment of the present invention in which the catheter (seen in FIG. IA) includes first and second pluralities of (circumferentially staggered) side holes that are spaced axially (longitudinally) along the catheter.

FIG. 21 is a perspective view of an alternate embodiment of the disclosure in which the catheter (seen in FIG. IA) includes a plurality of side holes arranged in a (generally) helical pattern.

FIG. 22 is a perspective view of an embodiment of the present invention in which the catheter (seen in FIG. IA) includes a plurality of (circumferentially staggered) balloon elements.

FIG. 23 is a transverse, cross-sectional view taken through line 23-23 in FIG. 22.

FIG. 24 is a perspective view of an alternate embodiment of the present invention in which the catheter (seen in FIG. 1) includes a plurality of balloon elements arranged in a (generally) helical pattern.

FIG. 25 is a perspective view of an alternate embodiment of the present invention in which the catheter (seen in FIG. 1) includes a plurality of (circumferentially staggered) side holes and a plurality of (circumferentially staggered) balloon elements.

FIG. 26 is a perspective view of an alternate embodiment of the present invention in which the catheter (seen in FIG. 1) includes a plurality of (circumferentially staggered) side holes and a plurality of balloon elements arranged in a (generally) helical pattern.

FIG. 27 is a perspective view of an alternate embodiment of the present invention in which the catheter (seen in FIG. 1) includes a plurality of side holes arranged in a (generally) helical pattern and a plurality of (circumferentially staggered) balloon elements.

FIG. 28 is a perspective view of an alternate embodiment of the present invention in which the catheter (seen in FIG. 1) includes a plurality of side holes arranged in a (generally) helical pattern and a plurality of balloon elements arranged in a (generally) helical pattern.

FIG. 29 illustrates use of the catheter during an endovascular procedure to treat a vascular abnormality.

FIG. 30 is a perspective view of an alternate embodiment of the catheter of the present invention including a pull wire configured to apply a torsional (twisting) force to the catheter.

FIG. 31 is a perspective view of an alternate embodiment of the catheter of FIG. 30 in which the pull wire includes a substantially helical (spiraled) distal segment and a (generally) linear proximal segment.

FIG. 32 is a perspective view of an alternate embodiment of the catheter of FIG. 30 including a plurality of pull wires.

FIG. 33 is a perspective view of an alternate embodiment of the catheter of FIG. 32.

FIG. 34 is a perspective view of an alternate embodiment of the catheter of FIG. 32.

FIG. 35 is a perspective view of an alternate embodiment of the catheter of FIG. 34.

FIG. 36 is a perspective view of an alternate embodiment of the catheter of FIG. 34.

FIG. 37 is a perspective view of an alternate embodiment of the catheter of FIG. 34.

FIG. 38 is a schematic representation of an alternate embodiment of the catheter of FIG. 1A shown in a first (initial, normal) configuration and including one or more steerable segments that are deflected by one or more pull wires.

FIG. 39 is a transverse cross-sectional view of the catheter of FIG. 38 taken along line 27-27.

FIG. 40 is a schematic representation of the catheter of FIG. 38 shown in a second (subsequent, deflected) configuration.

### DETAILED DESCRIPTION OF THE INVENTION

Referring now to the drawings wherein like reference numerals identify similar structures, element, and features, various embodiments of the presently disclosed systems and devices will be discussed.

The term “recoil and displacement”, as used herein refers to the phenomenon of catheter prolapse or displacement (slipping forward, back, or down, and out of the desired position) due to a counterforce against the catheter by the advancing wire, second catheter, or other, additional device.

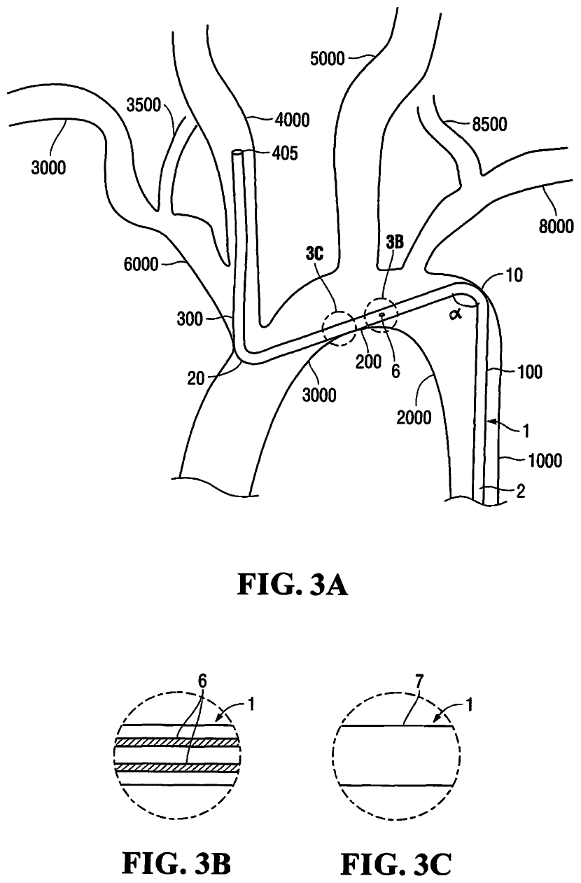

The catheters of the present invention, in some embodiments, use the anatomical fulcrum as an anti-kickback, anti-displacement support structure. Beyond the shaping of the catheter to allow said support, the catheter in some embodiments deploys a final element at the distal end to facilitate delivery of the distal end to the target area. The final element of the simplest embodiment of the invention is shown in FIG. 3A.

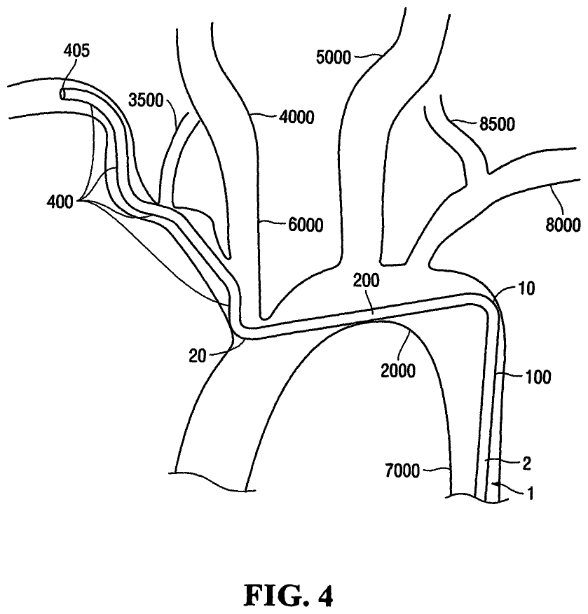

The final element in the preferred embodiment comprises two bends, three segments and one end hole. The final element may comprise one or more additional bends and one or more additional segments beyond those comprising the preferred embodiment. The final-element configuration is determined by the path the user of the catheter determines is necessary to deliver the distal end hole 405 to the target area as illustrated in FIG. 4. Additionally, although most commonly the second segment will rest on a vascular fulcrum, any segment, or in some cases multiple segments, may utilize a vascular anatomical structure for securement, to prevent unwanted kickback and displacement and prevents prolapse of the catheters or supplemental devices during delivery. Additionally, in some embodiments, the catheter has an additional circumferential balloon near its distal end hole, with at least one additional lumen to inflate and deflate said balloon positioned substantially within the wall of said catheter, in its intraluminal segment. Not pictured is a proximal branching of said additional lumen to its own proximal end hole at its own external termination device, said branching which occurs outside the patient's body, which is well known in the prior art. Inflation of said balloon is capable of completely occluding the ipsilateral common carotid artery proximal to the target stenosis, which is typically in the internal carotid artery, thereby allowing reversal of flow in said internal carotid artery when flow is allowed through the (main) catheter of the current invention. This flow can be active or passive. It can be aided in some cases by proximal aspiration, vacuum, or other pump mechanism. It can be aided in some cases by differential pressure between the target artery and a vein to which a circuit of flow is subsequently established. The vein can be accessed with a secondary (separate) catheter, and tubing can connect the proximal end of said (main) catheter with the proximal end of said secondary (separate) catheter. In some embodiments a regulator along said tubing can modulate flow rates as well. In some embodiments a filter along said tubing can filter the blood and remove embolic debris before returning it to the patient as well. In some embodiments flow reversal can also be augmented by infusion of fluids across and/or distal to said target lesion, as previously described with similar devices by the present inventor (Walzman). Such infusions can also act to decrease a known sump effect from the brain while flow is reversed across the lesion, thereby decreasing the potential for ischemia of the brain tissue that can result from said sump effect.

Now referring to FIG. IA, a medical device M (e.g., a catheter C) is disclosed that includes the tube 1 of the present invention. The tube 1 includes a proximal end hole 404 and the aforementioned distal end hole 405, which is positioned opposite to the proximal end hole 404. The catheter C is shown deployed in the aorta with distal end hole 405 terminating in an abnormal anatomical variation of the left carotid artery 500 referred to as a bovine arch. The device of this embodiment of the current invention has seven principal elements. The first three of the elements are bends, and four are segments of the tube. More particularly, first bend 10 connects segment one 100 to segment two 200 at a non-obtuse angle a, as measured as an angle from the proximal catheter tubing to the tubing of the second segment, in this example (e.g., such that the angle a. (FIG. IA) is defined between respective longitudinal axes Xi, Xii of segments 100, 200). It is envisioned, however, that segments 100, 200 may be configured and positioned so as to achieve any necessary or desired angle a. (e.g., depending upon the particular nature of the procedure being performed, spatial restrictions dictated by the patient's vasculature, etc.).

First bend 10 extends in a first direction and may be active or passive. A passive bend, as disclosed by the prior art, is a bend which is pre-formed by the use of a wire or a braid. A passive bend 10 has been treated in such a way prior to the introduction to the body that, if there are no other forces, it will form a desired (e.g., non-obtuse) angle. In order to deploy (insert), a tube must be straight, so there must be a force to straighten bend 10, such as a wire, a stiff inner or outer tube or combination, such that upon removal of said external force, the desired (e.g., non-obtuse) angle is formed. In other embodiments, any bend may be active or passive. In some embodiments, all bends are active (requiring) remote manipulation by a user such as by a pull (or push) wire attached to a catheter segment); in other embodiments all bends are passive; in yet other embodiments, bends may be a mix of active bends and passive bends.

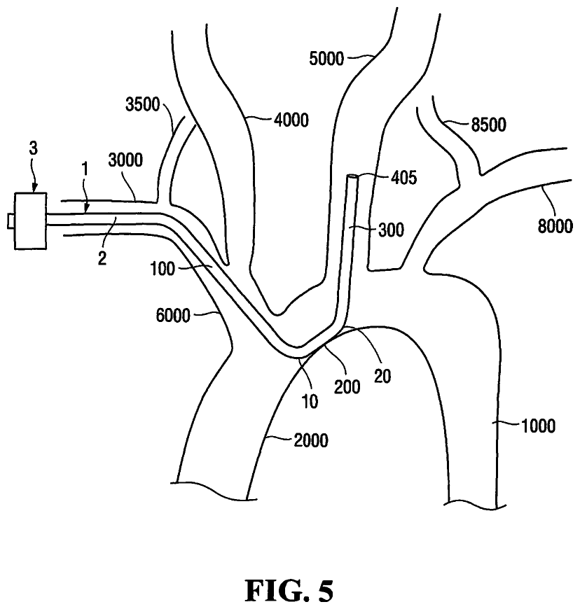

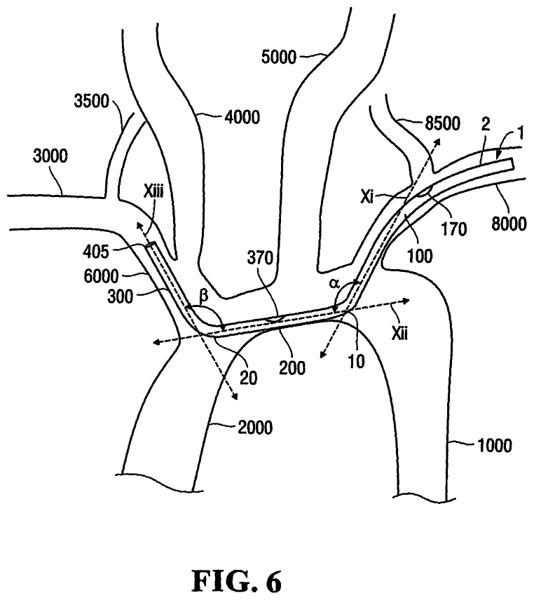

Other embodiments are adapted to access aortic arch 2000 through a vessel in the arm or, for example, from a radial artery, brachia! artery, axillary artery (or vein) (not shown), when such access may be preferred. In the embodiment depicted in FIG. 5, the catheter includes tube 1 having at least one operational (primary, working) lumen 2 that extends through the catheter from the proximal end hole 404 to the distal end hole 405. In FIG. 5, first segment 100 is shown accessing aortic arch 2000 through right subclavian artery 3000. FIG. 5 further illustrates tube 1 having second segment 200 disposed between first bend 10 and second bend 20, resting on the fulcrum of arch 2000 and third segment 300 deployed in the common carotid artery 5000. As seen in FIG. 6, it is envisioned that the second segment 200 may include an optional side hole 370 that is located between bends 10, 20. For example, it is envisioned that side hole 370 may be configured to allow for the passage of a supplemental medical device (e.g., a catheter, a deburring device, etc.) therethrough.

During the course of the endovascular procedure, it is envisioned that the second segment 200 may be moved off of (e.g., away from) the fulcrum of the arch 2000 and that the tube 1 may be repositioned such that the third segment 300 (or any other suitable segment of the tube 1) rests against the fulcrum of the arch 2000.

In a variant embodiment of FIG. 5, segment 100 of tube 1 is shown as having a “gentle curve” nearest to an external termination device 3 (e.g., a Luer lock) and at least two bends 10 and 20.

FIG. 6 illustrates a second segment 200 of tube 1 resting on aortic fulcrum 2000. Aortic fulcrum 2000 is accessed via an arm vessel or axillary artery or vein from the opposite arm illustrated in FIG. 5. In the embodiment of FIG. 6, tube 1 passes through left subclavian artery 8000. In the illustrated embodiment at least one side hole 170 is disposed proximal to left vertebral artery 8500. As seen in FIG. 6, the side hole 170 is formed in segment 100 and is located proximally of bend 10. To properly access the vasculature, it is envisioned that the segment 100 may be either (generally) linear, as seen in FIG. 4, for example, or that segment 100 may include a non-linear configuration defining a curvature, as seen in FIG. 6, for example. Second segment 200 rests on aortic fulcrum 2000 and extends from segment 100 such that the angle<i is obtuse. Second segment 200 further includes side hole 370 disposed proximal to aortic fulcrum 2000 and between bends 10, 20. Second bend 20 directs third segment 300 upward into innominate artery 6000 wherein end hole 405 is disposed. Third segment 300 extends from second segment 200 such that an P angle is defined between respective longitudinal axes Xii, Xiii of segments 200, 300. In the illustrated embodiment, segments 200, 300 are configured and positioned such that angle p is obtuse and such that second bend 20 extends in a second direction that is (generally) opposite to the first direction of first bend 10. It is envisioned, however, that segments 200, 300 may be configured and positioned so as to achieve any necessary or desired angle p (e.g., depending upon the particular nature of the procedure being performed, spatial restrictions dictated by the patient's vasculature, etc.).

As seen in FIG. 6, the side holes 170, 270 are arranged in a staggered pattern along a longitudinal axis (length) of the tube 1 such that the side holes 170,270 are spaced axially from each other. Although illustrated as being in (general) axial alignment in FIG. 6, it is also envisioned that the side holes 170, 270 may be staggered circumferentially as well, as discussed in further detail below.

The combination of a catheter that utilizes the inferior curve of the aortic arch as a vascular fulcrum with side holes, through which additional catheters can be passed, may further facilitate catheterization of bilateral vertebral and carotid arteries via a single access site in either arm.

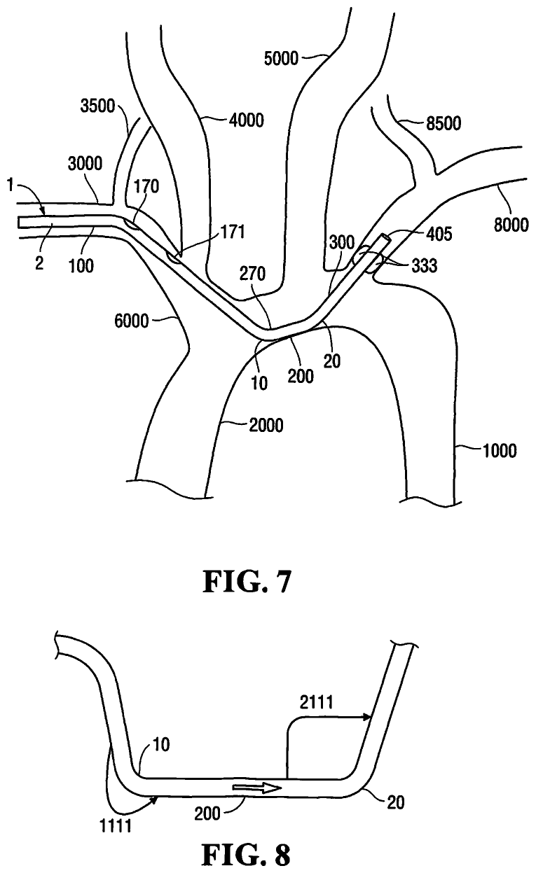

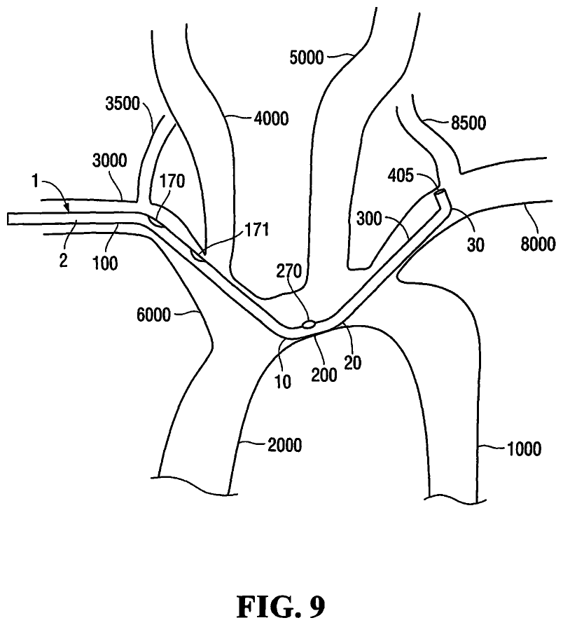

As depicted in FIG. 7, another embodiment of the catheter also accesses aortic arch 2000 through an arm vessel. In the embodiment of FIG. 7, the catheter has a tube 1 which includes lumen 2 and a first segment 100 that is configured to access aortic arch 2000 through the right subclavian artery 3000. FIG. 7 further illustrates tube 1 having side hole 270 disposed upon second segment 200 within aortic arch 2000 and side hole 170 disposed upon first segment 100 deployed within right subclavian artery 3000 proximal to the entry point of right vertebral artery 3500, and can further have an additional third side hole 171 proximal to the origin of the right common carotid artery. As discussed in connection with side holes 170, 270, it is envisioned that side hole 171 may be configured to allow for the passage of a supplemental medical device (e.g., a catheter, a deburring device, etc.) therethrough.

It should be noted that in embodiments containing more than one balloon, each balloon may optionally require a separate inflation lumen.

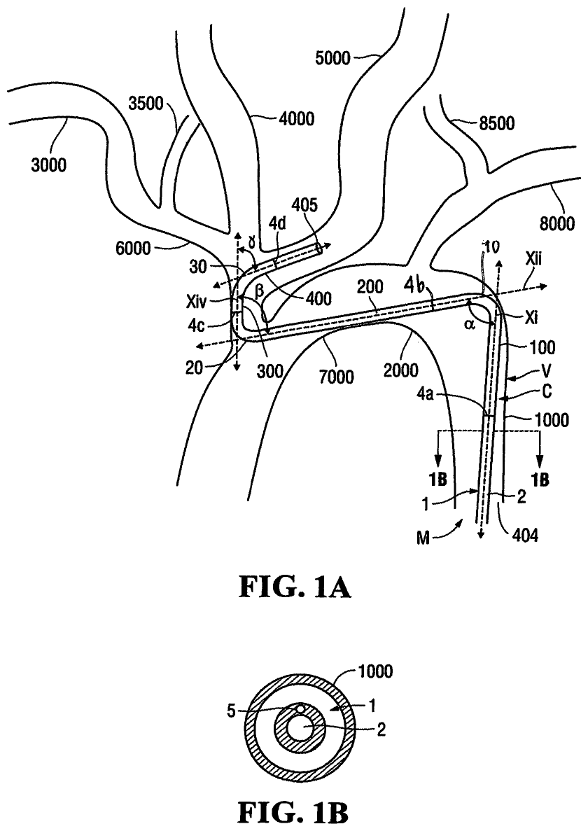

Other variants of catheter embodiments optionally include at least one valve 4. For example, as seen in FIG. IA, tube 1 may include a first valve 4a located within segment 100 proximally of bend 10; a second valve 4b located within segment 200 distally of bend and proximally of bend 20; a third valve 4c located within segment 300 distally of bend and proximally of bend 30; and/or a fourth valve 4d located within segment 400 distally of bend 30 and proximally of end hole 405. It is envisioned that each of the valves 4 may be configured to receive a medical device in sealed engagement. It is also envisioned that each of valves 4 may be biased towards a closed position to regulate blood flow through one or more segments 100, 200, 300, 400 of tube 1. Still other variants of catheter embodiments may optionally include at least one supplemental irrigation lumen 5 (FIG. 1B) substantially in the wall of tube 1, which may include an end hole terminating either inside or outside tube 1. It is envisioned that lumen 5 may be configured to communicate an irrigation fluid through tube 1 to help minimize clot formation in the exit region adjacent to a target area in the vasculature.

In an alternative embodiment, materials or techniques may be employed so as to achieve any desired configuration for bends 10, 20, 30. For example, materials may be chosen and techniques utilized such that bends 10, 20, 30 are obtuse, non-obtuse, or at approximately right angles. Such embodiments may include the incorporation of shape-memory metals or polymers. In addition or in the alternative, radiation may be focused on a point of tube 1 such that bend 10 is forced to adopt a desired, non-obtuse angle of segment two relative to the proximal segment one to position segment two over the fulcrum of aortic arch 2000.

Segment one 100 in some embodiments has a length of at least approximately 20 cm in length and an internal diameter of from approximately 0.1 French to approximately 30 French. In a preferred embodiment deployed transfemorally for access of the innominate arteries distal branches with a Type II and Type III arch, first bend 10 is deployed in the artery such that angle a is non-obtuse so as to orient segment two 200 for optimal positioning on the fulcrum of aortic arch 2000.

Segment two 200 in some embodiments measures at least approximately 3 cm in length and no more than approximately 35 cm in length in the preferred embodiment of FIG. 3A. Segment two 200 in some embodiments has an internal diameter of from approximately 0.1 French to approximately 30 French. Segment two 200 has a first end which terminated in first bend 10 and a second end which terminates in second bend 20. It is envisioned in some embodiments that segments 200, 300 may be configured and positioned such that angle lies substantially within the range of approximately 30 degrees to approximately 150 degrees.

Second bend 20 connects to segment three 300 of tube 1. Segment three 300 measures at least approximately 0.5 cm in length and has an internal diameter of from approximately 0.1 French to approximately 30 French. Segment three 300 has a first end which terminates in second bend 20 and connected to segment two 200 of tube 1, and a second end terminating at third bend 30.

Third bend 30 connects to segment four 400 (FIG. 4) of tube 1 and extends in a third direction different from the first direction (of first bend 10) and the second direction (of second bend 20). Segment 400 extends from second segment 300 such that an angle y (FIG. 1A) is defined between respective longitudinal axes Xiii, Xiv of segments 300, 400. In the illustrated embodiment, segments 200, 300 are configured and positioned such that angle y is approximately 90 degrees. It is envisioned, however, that segments 300, 400 may be configured and positioned so as to achieve any necessary or desired angle y (e.g., depending upon the particular nature of the procedure being performed, spatial restrictions dictated by the patient's vasculature, etc.). For example, it is also envisioned that segments 300, 400 may be configured and positioned such that angle y is acute or obtuse.

Segment four 400 in some embodiments measures at least approximately 0.5 cm in length and has an internal diameter of from approximately 0.1 French to approximately 30 French. Segment four 400 has a first end which terminates in third bend 30 and connected to segment three 300 of tube I, and a second end terminating at distal hole 405.

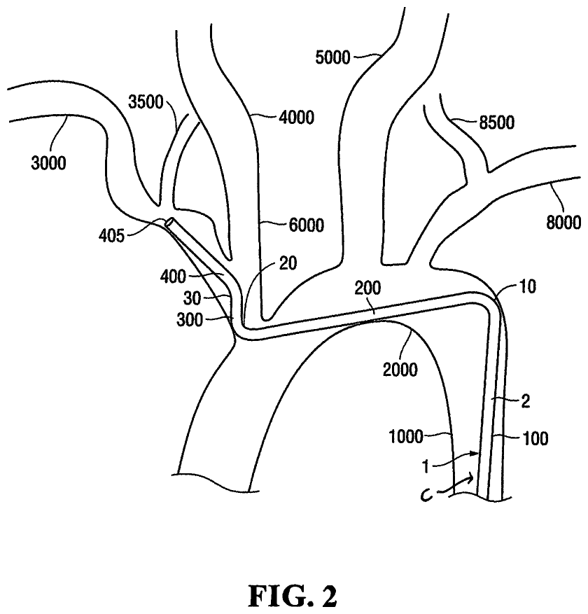

Now referring to FIG. 2, a catheter of the present invention is shown deployed in a Type III aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. Segment three 300 is shown in this example being upwardly deployed into innominate artery 6000, and segment four 400 extending upwardly from third bend 30 at an obtuse angle, relative to the catheter of segment three 300, and deployed distally in right subclavian artery 3000.

Now referring to FIG. 3A, the catheter of the present invention is shown deployed in a sample aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. In this embodiment, the first bend 10 (e.g., angle a) is shown as being non-obtuse. While this is a feature of this embodiment, a non-obtuse bend is not a limitation of the present invention, rather a shape of a particular embodiment which will allow the use of the arch of the aorta 2000 to prevent kick-back and displacement in particular anatomical scenarios. An obtuse bend will not allow the use of the arch of the aorta 2000 to prevent kick-back and displacement while obtaining transfemoral access to the right carotid artery in these select anatomical variants.

The middle segment 200 of the catheter can includes ridges 6 such as shown in FIGS. 3A and 3B. to, promote stability at the focal point 2000. Although shown as extending longitudinally (e.g., in (generally) parallel relation to axis Xii (FIG. IA), it is also envisioned that ridges 6 may extend transversely (e.g., in (generally) orthogonal relation to axis Xii (FIG. IA)). Additionally, while segment 200 is illustrated as including two ridges 6 in the illustrated embodiment, it should be appreciated that the number of ridges 6 may be increased or decreased in alternate embodiments without departing from the scope of the present disclosure. For example, embodiments in which segment 200 includes a single ridge 6 or three (or more) ridges 6 are also contemplated herein.

According to another embodiment, the middle segment two 200 is coated with an elastic material 7 (FIG. 3C) to deform adjacent to (e.g., atop) the fulcrum point 2000 for improved securement.

The various components of the described invention may be comprised of one or more materials. Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFB) and polyimide. Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or co-polyesters such as XYDAR® (Amoco) or VECTRA® (Hoechst Celanese).

According to some embodiments, the bends comprise a shape memory polymer (SMP). Shape memory polymers include, but are not limited to meth-acrylates, polyurethanes, blends of polystyrene and polyurethane, and PVC. According to some embodiments, the bends of the catheter comprises a shape memory alloy (SMA). Non-limiting examples of shape memory alloys include nickel-titanium (i.e., nitinol).

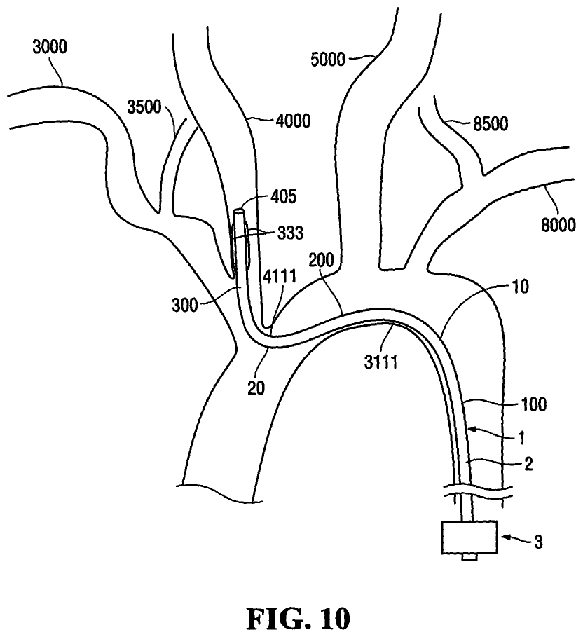

Now referring to FIG. 10 which discloses a preferred embodiment for the right carotid, the first segment 100 extends from the external termination device 3 (e.g., the aforementioned Luer lock) through first curve 3111 of bend 10. In the current embodiment, the distal region of the first segment 100, at first curve 3111, and extending into second curve 4111 of bend 20, which extends into the second segment 200 and curves in a substantially opposite direction to first curve 3111, are optimized to rest upon the lesser curve of the aortic arch, thereby providing support, and bracing the catheter within the blood vessel so as to inhibit (if not entirely prevent) recoil (e.g., kickback, prolapse, etc.) of tube 1 of the catheter and any additional (supplemental) medical devices that are subsequently passed through tube 1 into the distal vasculature, examples of which are discussed below. It should be noted that second segment 200 is bounded proximally by first bend 10 and first curve 3111, and distally by second bend 20 and second curve 4111.

In certain embodiments, first segment 100 of tube 1 may having an effective length (segment within the body) that lies substantially within the range of approximately 30 cm to approximately 70 cm, and second segment 200 of tube 1 may have an effective length that lies substantially within the range of approximately 4 cm to approximately 25 cm when used transfemorally for carotid bifurcation pathology. The tube 1 may include an outer diameter that lies substantially within the range of approximately 4 Fr to approximately 12 Fr for this application. The tube 1 additionally has at least one circumferential balloon 333 near (e.g., at or (generally) adjacent to) its distal end hole 405, which is optimized for atraumatic temporary occlusion of the common carotid artery during angioplasty and stenting, in order to create flow reversal across the lesion. The (primary working) lumen 2 of tube 1 includes an internal diameter sufficient to allow for the insertion and delivery of additional (e.g., supplemental) medical devices (such as balloons, hypotubes, wires, stents, etc.). The tube 1 may also include at least one additional lumen (e.g., the aforementioned irrigation lumen 5 (FIG. 1B), which extends within the wall of the tube 1 in (generally) parallel relation to the lumen 2 along all or a portion of the effective length of said tube 1 to facilitate inflation and deflation of said at least one circumferential balloon 333. It is envisioned that, in certain embodiments, bend 10 (e.g., first curve 3111) may lie substantially within the range of approximately 60 degrees to approximately 120 degrees, and that, in certain embodiments, the bend 20 (e.g., second curve 4111) may lie substantially within the range of approximately 65 degrees to approximately 130 degrees. As seen in FIG. 10, for example, bends 10, 20 curve in (generally) opposite directions. To facilitate insertion of tube 1 into the vasculature, it envisioned that a straight inner dilator may be used to substantially straighten said tube 1, as is well known in the prior art/field.

Like the prior art described above, the current invention relies on flow reversal across the lesion during angioplasty and stenting to minimize the risks ofthromboembolic ischemic complications during the procedure. However, whereas the cited prior art relies on a carotid open surgical cut-down, the current invention optimally uses percutaneous techniques. The current invention additionally, in the preferred transfemoral embodiment, utilizes vascular fulcrums for support of the devices, to reduce potential complications and risks. Furthermore, as previously described by the inventor of this application (Walzman), the current device additionally optionally utilizes infusion of fluid distal to the lesion during the procedure to aid in flow reversal across the lesion, while minimizing a sump effect from the brain that can contribute to ischemic complications. In order to accomplish this, these embodiments of the current invention optimally utilize a hypotube capable of irrigation, in addition to its role as an access rail for balloons mounted on their delivery catheters as well as stents mounted on their respective delivery catheters, and/or additional balloon catheters capable of irrigation as well. In other embodiments the current invention may deploy an additional temporary balloon to occlude the vessel distally.

The current invention also in some embodiments utilizes angioplasty balloons on catheters that can also irrigate, and or additional irrigation catheters. Additionally, the current invention in some embodiments utilizes a double balloon catheter, wherein one balloon is optimized for angioplasty and at least one additional balloon is optimized for atraumatic temporary balloon occlusion of a vessel. In this way, an angioplasty balloon can be advanced over a wire, the wire optionally having an inner lumen and distal end and/or side holes for irrigation, and the angioplasty balloon can be inflated across the lesion to dilate the stenosis, and then deflated. The occlusion balloon can be proximal or distal to the angioplasty balloon; in the preferred embodiment it is proximal. To reduce the number of exchanges necessary during the procedure, each of which can increase risks, the balloon can then optionally be advanced past the lesion and not removed. The second balloon temporary occlusion balloon can then be inflated distal to the lesion, further decreasing the potential for a sump effect of blood flow from the brain during the procedure.

Additional fluids can then be infused through the double balloon catheter, with egress ports optionally both proximal and optionally distal to the occlusion balloon, to aid in flow reversal across the lesion proximally, and prevent clot formation distal to the occlusion balloon during balloon occlusion. The balloon, a conventional single angioplasty balloon, and/or the irrigation catheter 9300 (or hollow wire capable of irrigation) can further optionally have a detachable hub. The optional detachable hub can have pressure-mounted design or a threaded-screw design, or others.

Threaded screw designs can include a thread on the inside of the detachable hub and a corresponding opposite thread on the outside of the proximal end of the catheter, or alternatively the thread can be on the outside of the distal side of the hub and on the inside of the proximal end of the catheter. This removable hub (not shown) will allow these devices to be used as a rail (like a wire) to deliver additional catheters, such as an angioplasty balloon mounted catheter or a stent delivery catheter, both in an “over-the-wire” configuration and in a “rapid exchange” configuration, by allowing the additional catheters to be loaded over the proximal end of these catheters after the hub is detached. The catheters can additionally have in some embodiments valves in order to prevent deflation of a temporary occlusion balloon during hub detachment. The hub can be re-attached to allow continuation of fluid delivery and/or balloon deflation when desired.

All described catheters and wires can have tapered or non-tapered distal ends.

Stents can be self-expanding, balloon expanded, or a hybrid.

The current invention can include in some embodiments a plug or balloon to occlude the external carotid artery, to further ensure flow is reversed across the stenosis in the internal carotid artery during angioplasty and stenting. The plug or balloon can be mounted on a wire or catheter, can be detachable or non-detachable, can be retrievable or non-retrievable, and/or can be permanent or temporary. One example of a temporary detachable plug is a biodegradable hydrogel plug, which the body can recanalize.

In an embodiment, the device of the present invention further comprises at least one vascular plug, capable of obstructing collateral flow from a branch such as the external carotid artery. The plug is preferably located between at least one circumferential balloon and a vascular blockage to further ensure flow is reversed at the obstruction during angioplasty and stenting. It should be noted that in one embodiment, a patient's body will break down the plug and restore flow in a vascular branch over a set period of time.

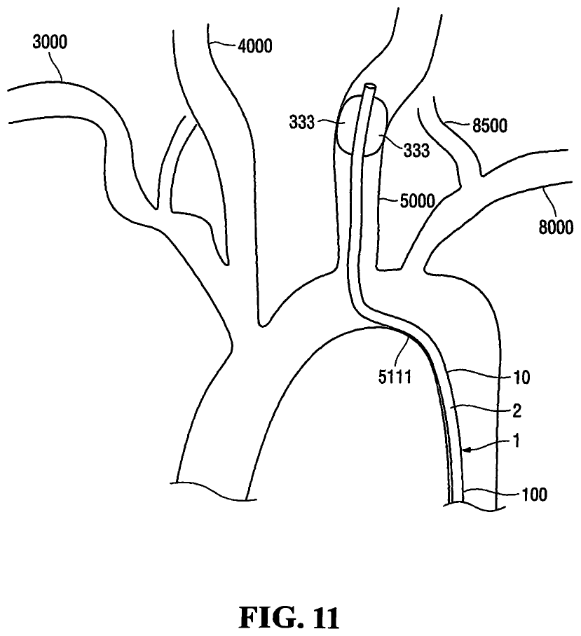

Now referring to FIG. 11 which discloses an embodiment for catheter placement in the left carotid. The left carotid and right carotid arteries are sized in accordance with the anatomical dimensions of the arteries. The dimensions vary from patient to patient but are readily determinable. Accordingly, embodiments intended for left carotid use will be sized as described above for the right carotid artery, with adjustments for these variations.

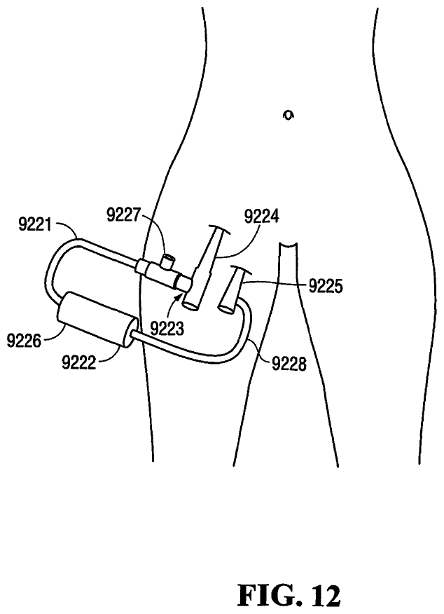

Now referring to FIG. 12 which illustrates the external elements of embodiments of the present invention, and more particularly illustrating that approach can be right femoral artery and/or left femoral artery (right illustrated). Radial, brachia!, or axillary arterial access, and other percutaneous ports of access, can be used as well. For example, to inhibit blood loss, the external elements include some or all of filter 9222, Y-connector 9223, external termination device of arch-fulcrum catheter such as a Luer Lock element 9224, venous sheath 9225, flow regulator 9226, and stopcock 9227; and tube elements 9221 and 9228. During use of the medical device 14, blood flow may be reversed in the target blood vessel V (e.g., artery) such that blood flows through (and from) the medical device M. This blood can either be discarded (if the volume is negligible) or re-circulated to the patient. In those instances of recirculation, prior to returning to the patient (e.g., via the venous sheath 9225), it is envisioned that blood may be passed through the filter 9222 to remove debris. When necessary or desirable, directing blood flow through the filter 9222 and the venous sheath 9225 may create a passive flow mechanism in that blood flow may be directed from a (higher pressure) artery, through the medical device M, and into a (lower pressure) vein. Should higher velocities and/or volumes of flow reversal be necessary or desirable, such as, for example, when the lumen 2 extending through the tube 1 is partially obstructed (e.g., via stent 8971, irrigation catheter 9300, etc.), it is envisioned that the flow velocity and/or the flow rate may be augmented using a pump or a vacuum.

Alternatively, venous sheath 9225 can be used in any vein of sufficient size. It should also be noted that the flow regulator 9226 can be any one previously disclosed by the prior art: a wheel on a ramp (like a standard), or can involve routing blood through a higher or lower resistance path. Alternatively, the regulator can be active, utilizing pumps, artificial pressure gradients, vacuums, or other mechanisms that can increase flow through a narrow path when desired, thereby allowing a smaller sized delivery catheter to still effect flow reversal during device delivery, thereby reducing potential for access site complications, and increasing available ports of entry.

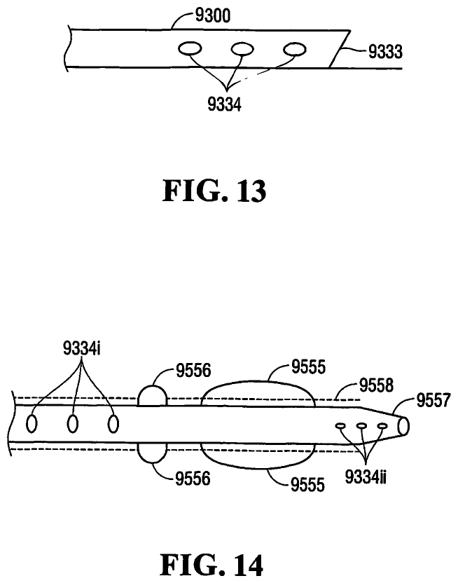

FIG. 13 illustrates one example of a (first) supplemental medical device that is configured for insertion into the blood vessel V through the catheter C. More specifically, the supplemental medical device is shown as the aforementioned irrigation catheter 9300, which includes a distal end 9333 and a plurality of irrigation ports 9334.

FIG. 14 depicts an embodiment of the irrigation catheter 9300 of FIG. 13, further including angioplasty balloon element 9555, occlusion balloon element 9556, and irrigation ports 9334. In the illustrated embodiment, the irrigation catheter 9300 includes a first plurality of ports 9334i that are located proximally of the occlusion balloon element 9556 and a second plurality of ports 9334ii that are located distally of angioplasty balloon element 9555. As seen in FIG. 14, it is envisioned that the irrigation catheter 9300 may include a tapered distal tip 9557 and that the occlusion balloon element 9556 may include a transverse cross-sectional dimension (e.g., a diameter) less than that of the angioplasty balloon element 9555. A still further embodiment also includes a “peel away sheath” 9558 to protect the access artery from the balloons 9555 and/or 9556, and the balloons 9555 and/or 9556 from the access artery and tissue, during insertion of the tube 1 and the balloons 9555, 9556. The peel-away-sheath 9558 can be very thin, and the tube 1 can optionally have a slightly larger outer diameter to prevent leakage around it after the peel-away sheath 9558 is removed after the balloons 9555 and/or, 9556 are positioned intravascularly.

In a still further embodiment, the disclosed medical device M further includes at least one of series of angioplasty balloons and/or stent delivery catheters with removable hubs and/or side ports. The series can be delivered over each other, such that a first delivery wire “rail” crosses a lesion. Then an angioplasty balloon is inflated, with flow reversed, optionally aided by active pumps or similar. As such, the current invention can have the hub and side port of the angioplasty balloon be removable. The method simply requires that the user advance the balloon, after angioplasty inflation and subsequent deflation, slightly past a target blockage. Then the user slides the next balloon catheter, or the stent catheter, over the balloon catheter. In the prior art, systems require exchanging the balloon catheter for another larger balloon or the stent. This maneuver enhances risk to patients; for example, the wire can move, the time for the procedure be increased, and/or an increased loss of blood can occur.

In some embodiments, the angioplasty balloon catheter is further capable of delivering fluid, which can be delivered distal to the blockage and/or across the blockage. Thereby, any potential “sump effect” of blood⋅flow diversion from the distal tissue is reduced, while flow is reversed across the blockage.

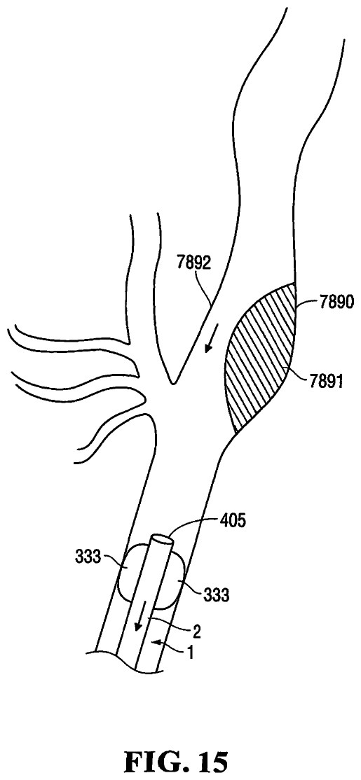

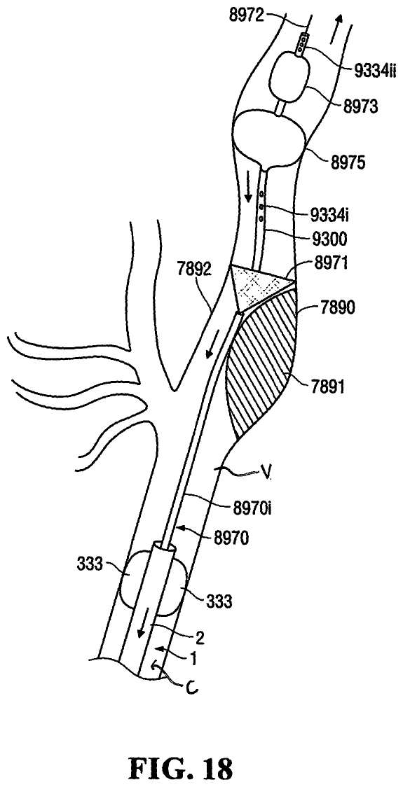

Referring now to FIGS. 15 through 18, it should be noted that the devices of present invention in some embodiments are capable of deployment of multiple supplemental medical (therapeutic) devices (e.g., the delivery catheter 8970 or hypotube 8970i, the fluid delivery (irrigation) catheter (hypotube) 8974, the irrigation catheter 9300, etc.) into a narrowed artery lumen 7892 (FIG. 15) through the operational lumen 2 of the tube 1 of the catheter (catheter C). For example, as illustrated and described herein, one or more supplemental medical devices may be delivered into the blood vessel V over delivery wire 8972 and either through or over fluid delivery (irrigation) catheter (hypotube) 8974 and/or irrigation catheter 9300. Irrigation catheter 9300 may include optional, multiple fluid-delivery ports 9334, as discussed in connection with the embodiment of the irrigation catheter 9300 seen in FIG. 14. Ports 9334 may be disposed proximally to temporary occlusion balloon element 8975 and/or distally to delivered angioplasty balloon 8973. In the embodiment of the disclosure seen in FIG. 18, for example, both balloon elements 8973 and 8975 are mounted on irrigation catheter 9300 distally of stent 8971, with the balloon element 8973 being located distally of the balloon element 8975. Stent element 8971 may also be delivered over the exterior of irrigation. catheter 9300 for simultaneous deployment. It should be further noted that the present invention in some embodiments implements a balloon-guide catheter (or sheath) capable of occluding the target CCA. As indicated in FIG. 18, when the device is deployed, fluid flow distal to the temporary occlusion balloon 8975 is static or flows distally, whereas proximally to temporary occlusion balloon 8975, fluid flows proximally through stent 8971, then through tube 1 of catheter C. In a preferred embodiment, delivery catheter 8970 (which is configured as hypotube 8970i in the embodiment of FIG. 18) is dimensioned sufficiently relatively smaller in diameter than the inner diameter of tube 1 to allow proximal blood to flow through to tube 1, while having a sufficient inner diameter to deliver over irrigation catheter 9300.

In an alternative embodiment, the present invention relates generally to endovascular devices and more particularly to specifically using a shaped support catheter and a hypotube in lieu of a wire to shape catheters. More particularly, the device uses hypotubes, and related elements to obviate the need for open surgical cutdowns of the common carotid artery (CCA) with a carotid stent, using a flow reversal loop system for embolic protection, while also employing a percutaneous technique and novel carotid access devices which use anatomical fulcrums for added support.

Additionally with respect to the embodiments in which one or more of the disclosed devices includes (or is configured as) a hypotube, the present invention combines direct carotid-artery access with rigorous blood flow-reversal, in order to protect the brain from embolic debris when introducing interventional devices into the carotid artery. Disclosed is a medical device capable of treating vascular blockages, more particularly a hypotube having at least one lumen extending from a proximal port to a distal end hole, capable of delivering additional medical devices, and at least one distal, circumferential balloon capable of temporary occlusion of native flow in a vessel near its distal end hole upon inflation. The disclosed hypotube is capable of delivering a second balloon for angioplasty, and at least one stent.

While other inventions of the inventor of the present application (Walzman) have disclosed the combined use of a wire for curving tubes, and a stent delivery catheter, the present invention discloses a hypotube to perform both of these functions. This configuration eliminates at least one element, thus simplifying the system/device/method, and reducing the possibility of failure. Additionally, by replacing the wire and delivery catheter with a hypotube, the hypotube will be smaller that the combination of those two, thus allowing access to smaller vessels.

As mentioned above, in certain embodiments, it is envisioned that the delivery catheter 8970 may be configured as (or may be replaced by) hypotube 8970i. For example, in FIG. 16, the narrowed internal carotid-artery lumen 7892 is shown as being accessed by hypotube 8970i, which is configured to deliver stent 8971. FIG. 16 further illustrates (blood) flow direction by arrows. It is envisioned that hypotube 8970i may also be configured to deliver a fluid therethrough to a target area with the vasculature.

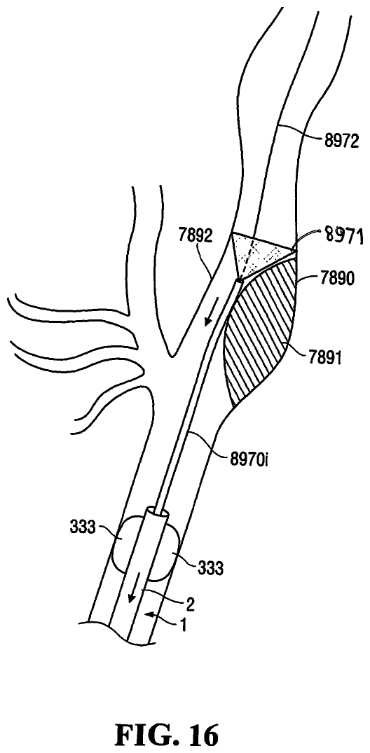

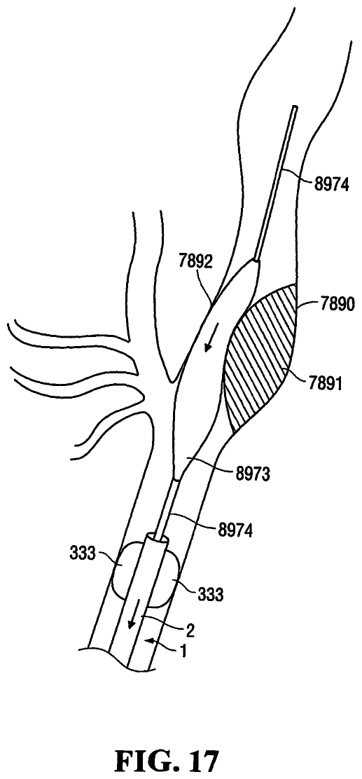

In an alternate embodiment, FIG. 17 illustrates the narrowed internal carotid-artery lumen 7892 of FIG. 15. In this embodiment, angioplasty occlusion balloon 8973 is disposed upon fluid delivery (irrigation) catheter (hypotube) 8974, the direction of blood flow again being shown by arrows. The fluid delivery (irrigation) catheter (hypotube) 8974 is configured for insertion into the vasculature through tube 1 of the catheter.

In an alternate embodiment, FIG. 18 illustrates the narrowed internal carotid-artery lumen 7892 of FIG. 15, further depicting delivery catheter 8970, which is configured as hypotube 8970i, and irrigation catheter 9300. It is envisioned that hypotube 8970i and irrigation catheter 9300 may be connected to each other (e.g., so as to form a single structure). It is also envisioned that hypotube 8970i and irrigation catheter 9300 may be formed as separate, discrete structures (e.g., such that irrigation catheter 9300 is insertable into the blood vessel V through hypotube 8970i).

In FIG. 18, irrigation catheter 9300 is shown as having the aforementioned fluid-delivery ports 9334, which are an optional feature of the structure. More specifically, the first plurality of fluid-delivery ports 9334i are disposed on the irrigation catheter 9300 proximally to temporary occlusion balloon element 8975 and the second plurality of delivery portions 9334ii are disposed on the irrigation catheter 9300 distally to balloon element 8973. As seen in FIG. 18, balloon elements 8973, 8975 are spaced longitudinally (axially) from each other along the length of irrigation catheter 9300; additionally, stent 8971 is also mounted on the exterior of hypotube 8970i. To facilitate delivery of the irrigation catheter 9300 in the manner depicted in FIG. 18, for example, hypotube 8970i includes a diameter allowing balloon elements 8973 and 8975 to pass therethrough. Additionally, as seen in FIG. 18, it is envisioned that stent element 8971 may be mounted on the outer surface of hypotube 8970i. Alternatively, it is envisioned that stent element 8971 may be mounted on the outer surface of the irrigation catheter 9300.

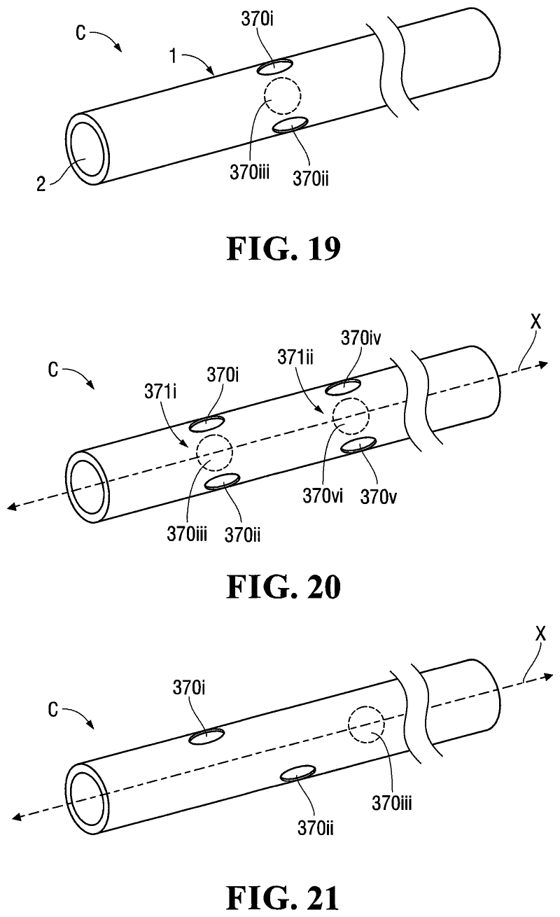

FIG. 19 illustrates an alternate embodiment of the catheter C in which the tube 1 includes a plurality of side holes 370 that are in communication with the operational lumen 2, each of which is configured to allow for the passage of a supplemental medical device (e.g., a catheter, a deburring device, etc.) therethrough and into the blood vessel V (FIG. IA). The side holes 370 are substantially similar (if not identical) to the aforedescribed side holes 170, 270 (FIG. 6) and are arranged in a staggered pattern along a circumference of the tube 1. In the particular embodiment illustrated, for example, the catheter C includes three side holes 370i, 370ii, 370iii that are separated by an angular (circumferential) distance of (approximately) 120° such that the side holes 370i, 370ii, 370iii are spaced (approximately) equidistant from each other. It should be appreciated, however, that the number of side holes 370 may be increased or decreased and/or the angular distances can be varied, in alternate embodiments without departing from the scope of the present disclosure. For example, the present disclosure also contemplates⋅embodiments in which the catheter C may include two side holes 370i, 370ii that are separated by an angular distance of (approximately) 180° (e.g., such that the side holes 370i, 370ii are positioned in (generally) diametric opposition), four side holes 370i-370iv that are separated by an angular distance of (approximately) 90°, etc. It is also envisioned that the side holes 370 may be positioned such that the circumferential spacing between adjacent side holes 370 varies. For example, it is envisioned that the side holes 370i, 370ii may be separated by a first angular distance, that the side holes 370ii, 370iii may be separated by a second angular distance, and that the side holes 370i, 370iii may be separated by a third angular distance, wherein at least one of the first angular distance, the second angular distance, and the third angular distance is unequal to the others (e.g., the side holes 370i, 370ii may be separated by an angular distance of (approximately) 90°, the side holes 370ii, 370iii may be separated by an angular distance of (approximately) 90°, and the side holes 370i, 370iii may be separated by an angular distance of (approximately) 180°). Other angular distance variations are also contemplated.

In the particular embodiment seen in FIG. 19, the side holes 370i, 370ii, 370iii are circumferentially aligned (e.g., positioned in (general) alignment along the circumference of the tube 1) such that the side holes 370i, 370ii, 370iii are arranged in a band 370. FIG. 20 illustrates another embodiment, however, in which the catheter C includes a first plurality of side holes 370i, 370ii, 370iii that are circumferentially aligned and arranged in a first band 371i and a second plurality of side holes 370iv, 370v, 370vi that are circumferentially aligned and arranged in a second band 371ii that is spaced axially (longitudinally) from the first band 371i along a longitudinal axis X (length) of the catheter C. Additional bands of side holes could be provided.

FIG. 21 illustrates another embodiment of the catheter C in which the side holes 370i, 370ii, 370iii are arranged in a staggered pattern where they are spaced both circumferentially and axially from each other along the longitudinal axis X such that the side holes 370i, 370ii, 370iii are arranged in a (generally) helical pattern.

It should be appreciated that the side holes could be of different sizes⋅and/or shapes than those shown in the drawings. Different axial (longitudinal) and/or radial distances are also contemplated.

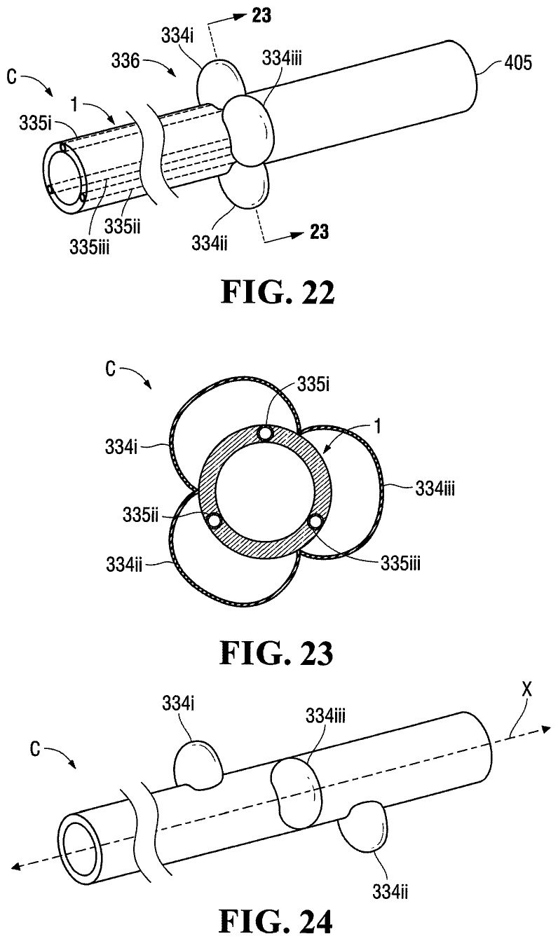

FIGS. 22 and 23 illustrate another embodiment of the catheter C, which includes a plurality of balloon elements 334 that are located proximally of the distal end hole 405 of the tube 1. The balloon elements 334 are substantially similar (if not identical) to the aforedescribed balloon element(s) 333 (FIG. 7) and are arranged in a staggered pattern. While described in connection with the catheter C, it should be appreciated that the balloon elements 334 and the various arrangements thereof discussed below may be applied to any of the catheters, hypotubes, etc., described herein, whether intended for therapeutic purposes or to facilitate the insertion and/or placement of another medical device (e.g., in the context of a delivery catheter).

In the particular embodiment illustrated in FIGS. 22 and 23, the catheter C includes three balloon elements 334i, 334ii, 334iii that are staggered along the circumference of the tube I such that the balloon elements 334i, 334ii, 334iii are separated by an angular (circumferential) distance of (approximately) 120°, whereby the balloon elements 334i, 334ii, 334iii are spaced (approximately) equidistant from each other. It should be appreciated, however, that the number of balloon elements 334 may be increased or decreased, and/or the circumferential distance may be different, in alternate embodiments without departing from the scope of the present disclosure. For example, the present disclosure also contemplates embodiments in which the catheter C may include two balloon elements 334i, 334ii that are separated by an angular distance of (approximately) 180° (e.g., such that the balloon elements 334i, 334ii are positioned in (generally) diametric opposition), four balloon elements⋅334i-334iv that are separated by an angular distance of (approximately) 90°, etc. It is also envisioned that the balloon elements 334 may be positioned such that the circumferential spacing between adjacent balloon elements 334 varies. For example, it is envisioned that the balloon elements 334i, 334ii may be separated by a first angular distance, that the balloon elements 334ii, 334iii may be separated by a second angular distance, and that the balloon elements 334i, 334iii may be separated by a third angular distance, wherein at least one of the first angular distance, the second angular distance, and the third angular distance is unequal to the others (e.g., the balloon elements 334i, 334ii may be separated by an angular distance of (approximately) 90°, the balloon elements 334ii, 334iii may be separated by an angular distance of (approximately) 90°, and the balloon elements 334i, 334iii may be separated by an angular distance of (approximately) 180°).

The balloon elements 334i, 334ii, 334iii are configured for selective, independent inflation (e.g., via separate inflation lumens 335i, 335ii, 335iii, respectively). In various methods of use, it is envisioned that one or more of the balloon elements 334 may be inflated to facilitate controlled deflection of the catheter C within the blood vessel V (FIG. IA). For example, inflation of the balloon 334i will deflect the catheter C in a (first) direction, inflation of the balloon 334ii will deflect the catheter C in a (second) direction, and inflation of the balloon 334iii will deflect the catheter C in a (third) direction. It is envisioned that one or more of the balloon elements 334i, 334ii, 334iii may be inflated simultaneously to facilitate deflection in a plurality of directions. It is also envisioned that each of the balloon elements 334i, 334ii, 334iii may be inflated simultaneously (e.g., to center the catheter C within the blood vessel.

In the particular embodiment seen in FIGS. 22 and 23, the balloon elements 334i, 334ii, 334iii are circumferentially aligned (e.g., positioned in (general) alignment along the circumference of the tube 1) such that the balloon elements 334i, 334ii, 334iii are arranged in a band 336. Additional bands of balloons could be provided. FIG. 24 illustrates another embodiment, however, in which the balloon elements 334i, 334ii, 334iii are arranged in a staggered pattern where they are spaced both circumferentially and axially from each other along the longitudinal axis X such that the balloon elements 334i, 334ii, 334iii are arranged in a (generally) helical pattern.

It should be appreciated that the balloon elements could be of different sizes and/or shapes than those shown. Different axial (longitudinal) distances are also contemplated.

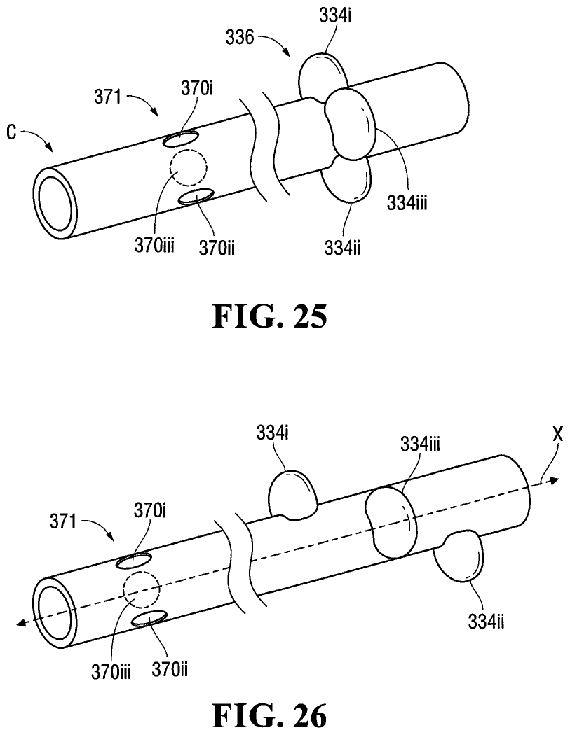

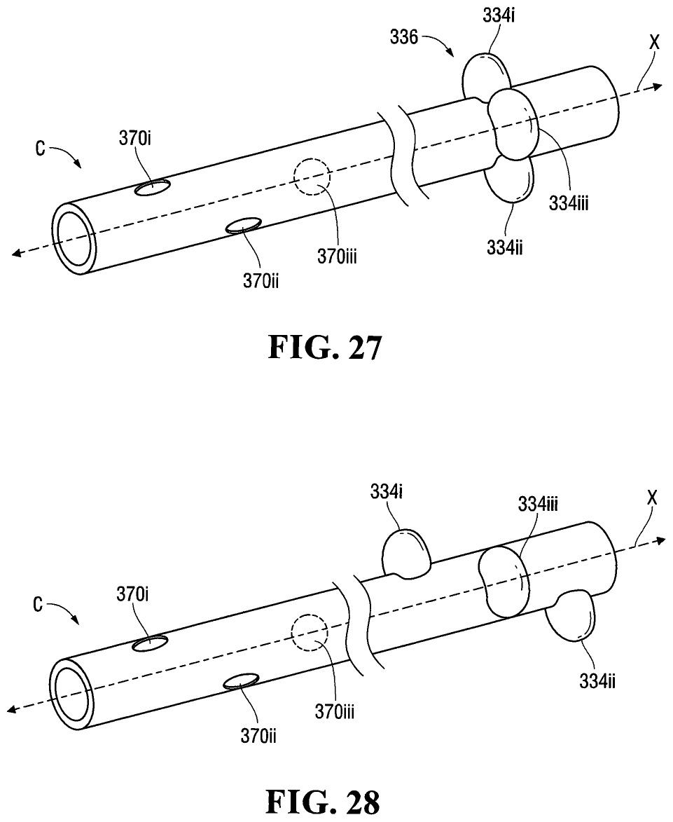

With reference now to FIGS. 25-28, several additional embodiments of the catheter Care illustrated that include both the side holes 370 (FIGS. 19-21) and the balloon elements 334 (FIGS. 22-24) in various arrangements. For example, FIG. 25 illustrates an embodiment of the catheter C in which the side holes 370i, 370ii, 370iii are circumferentially aligned and arranged into the (first) band 371 and the balloon elements 334i, 334ii, 334iii are circumferentially aligned and arranged into the (second) band 336. FIG. 26 illustrates another embodiment of the catheter C in which the side holes 370i, 370ii, 370iii are circumferentially aligned and arranged into the (first) band 371 and the balloon elements 334i, 334ii, 334iii are spaced both circumferentially and axially from each other along the longitudinal axis X of the catheter C such that the balloon elements 334i, 334ii, 334iii are arranged in a (generally) helical pattern. FIG. 27 illustrates another embodiment of the catheter C in which the side holes 370i, 370ii, 370iii are spaced both circumferentially and axially from each other along the longitudinal axis X of the catheter C such that the side holes 370i, 370ii, 370iii are arranged in a (generally) helical pattern and the balloon elements 334i, 334ii, 334iii are circumferentially aligned and arranged into the band 336. FIG. 28 illustrates another embodiment of the catheter C in which the side holes 370i, 370ii, 370iii and the balloon elements 334i, 334ii, 334iii are spaced both circumferentially and axially from each other along the longitudinal axis X of the catheter C such that the side holes 370i, 370ii, 370iii and the balloon element 334i, 334ii, 334iii are each arranged in a (generally) helical pattern. It should be appreciated that variations of the staggered side holes and balloon elements other than those shown could be provided, e.g., a different number, different angular spacing, different axial spacing, different sizes, different shapes, etc.

The single or multiple, e.g., staggered, balloons can be on the same catheter as the catheter having single or multiple, e.g., staggered, side holes or alternatively can be a separate outer catheter positioned over the catheter having the side holes(s).

During the course of an endovascular procedure, upon inserting and securing the catheter C within the blood vessel V (e.g., via inflation of one or more of the balloon element(s) 334), one or more supplemental medical (therapeutic) devices (e.g., the delivery catheter 8970 (FIG. 16), the hypotube 8970i (FIG. 18), the fluid delivery (irrigation) catheter (hypotube) 8974 (FIG. 7), the irrigation catheter 9300 (FIG. 13), a deburring device, etc.) may be inserted into the, catheter C. Thereafter, depending upon the location of the target site within the blood vessel V (e.g., a lesion, a branch of the blood vessel V, etc.), one of the side holes 370 is selected and the supplemental medical device(s) are passed therethrough such that the supplemental medical device(s) extend into the blood vessel V through the elected side hole 370. The staggered arrangement of the side holes 370 (circumferentially and/or axially spaced as described above) increases access to the target site and reduces (if not entirely eliminates) rotational manipulation of the catheter C that may be otherwise required to properly orient the supplemental medical device(s) relative to the target site, thereby reducing the complexity of the endovascular procedure.

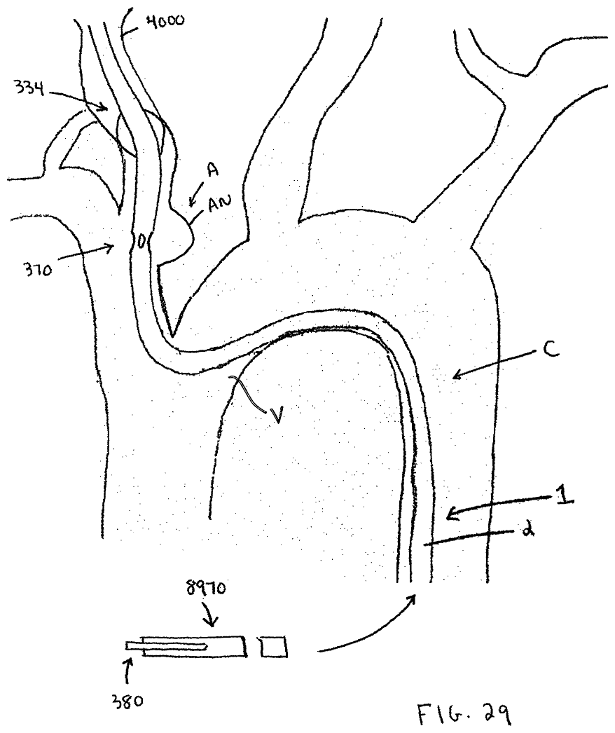

With reference now to FIG. 29, an endovascular procedure is illustrated in which the catheter C is utilized to treat a vascular anomaly (abnormality) A (e.g., an aneurysm AN, a lesion, a fistula, a rupture, or any other such malformation). Although illustrated as being located in the right carotid artery 4000, it should be appreciated that the specific location of the vascular abnormality A may vary and that the catheter C may be utilized to treat one or more vascular abnormalities A in a variety of locations within the patient's vasculature. Additionally, it should be appreciated that the endovascular procedure illustrated in FIG. 29 may be performed using any of the embodiments of the catheter C described herein (e.g., depending upon the particular nature of the vascular abnormality A being treated, the location of the vascular abnormality A, the requirements of the endovascular procedure, the particular anatomy of the patient's vasculature, etc.).

Initially, the catheter C is inserted into the patient's vasculature and is advanced therethrough (e.g., using fluoroscopy or any other suitable visualization method) until the side holes 370 are (generally) aligned with the vascular abnormality A. The catheter C is then secured using one or more of the balloon element(s) 334 (e.g., via the communication of fluid through the inflation lumens 335 (FIGS. 22, 23). If necessary, the catheter C can then be rotated (e.g., slightly rotated due to the provision of multiple side holes) to improve rotational alignment between one of the side holes 370 and the neck of the aneurysm AN and thereby facilitate the delivery of one or more supplemental medical (therapeutic) devices (e.g., the delivery catheter 8970 (FIG. 16), the hypotube 8970i (FIG. 18), the fluid delivery (irrigation) catheter (hypotube) 8974 (FIG. 17), the irrigation catheter 9300 (FIG. 13), a deburring device, etc.) into the aneurysm AN through the operational (primary, working) lumen 2 of the tube 1 and the side hole 370 aligned with the neck of the aneurysm AN. Thereafter, an embolic device 380 including one or more embolic agents, medications, synthetic materials, etc., may be delivered into the aneurysm AN through the supplemental medical (therapeutic) device (e.g., the delivery catheter 8970) extending through the selected side hole and the catheter C to treat the aneurysm AN.

During the particular procedure illustrated in FIG. 29, the catheter C may be positioned or braced against the patient's vasculature (e.g., against an inner wall of one of the patient's blood vessels) in the manner discussed above to thereby support the catheter C and inhibit (if not entirely prevent) recoil (e.g., kickback, prolapse, etc.) of the catheter C, the supplemental medical (therapeutic) device (e.g., the delivery catheter 8970), and/or the embolic device 380. For example, as discussed above in connection with FIG. 10, the catheter C may be oriented and braced against the lesser curve of the patient's aortic arch.

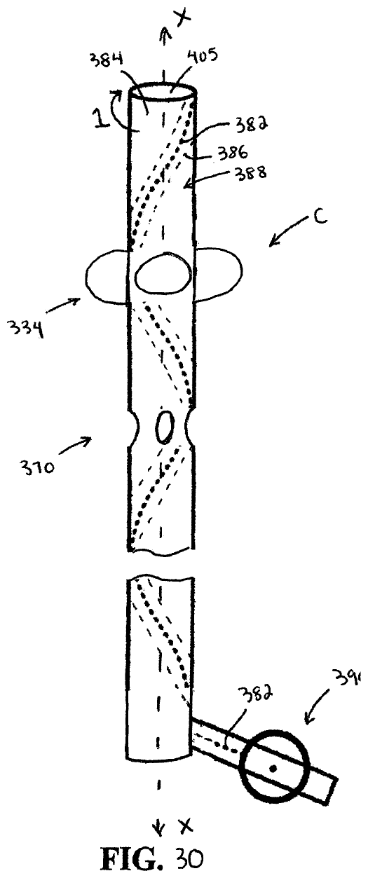

With reference now to FIG. 30, it is also envisioned that the catheter C may include one or more pull (or push) wires 382 that are connected (secured, anchored) thereto. The pull wire(s) 382 facilitate the selective application of a torsional (twisting) force to the catheter C to rotate the catheter C (along all or a portion of the length thereof) and, thus, the side hole(s) 370, to vary the rotational position of the side hole(s) 370 and improve rotational alignment with the vascular abnormality A. The pull wire(s) 382 extend in non-parallel relation to the longitudinal axis X of the catheter C. In the particular embodiment illustrated, for example, the catheter C includes a single pull wire 382 that is wound helically (spiraled) about the longitudinal axis X. It should be appreciated, however, that the number of pull wires 382 may be varied in alternate embodiments without departing from the present disclosure (e.g., it is envisioned that the catheter C may include two pull wires 382, three pull wires 382, etc.), as discussed in further detail below.

It is envisioned that the pull wire(s) 382 can fully or partially extend about the longitudinal axis X, i.e., extend 360 degrees, less than 360 degrees or greater than 360 degrees (more than one spiral). The pull wires can be fully or partially embedded in a wall of the catheter.

In some embodiments, the pull wire(s) 382 can be straight in part or most of the catheter C and substantially spiral in only part of its length. In some embodiments, the spiraling portion of the pull wire(s) 382 extends until at or near the distal end hole 405 of the catheter C. The spiraling of the pull wire(s) 382 can be configured so rotation occurs in a desired segment of the catheter C, for example, to rotate the orientation of the side hole(s) 370 as desired.

FIG. 31 illustrates another embodiment of the disclosure in which the pull wire(s) 382 include a (first) distal segment 382a that extends in non-parallel relation to the longitudinal axis X of the catheter C and a (second) proximal segment 382b that extends in (generally) parallel relation to the longitudinal axis X. More specifically, the distal segment 382a includes a curved (non-linear) configuration that is wound helically (spiraled) about the longitudinal axis X (e.g., at or adjacent to the distal end hole 405 of the catheter C) and the proximal segment 382b includes a (generally) linear configuration.

It is envisioned that the catheter C may define an overall length sufficient to allow for rotational deflection of the catheter C without any significant (substantial) kinking, binding, or other such undesirable deformation. For example, in certain embodiments, it is envisioned that the catheter C may define an overall length that lies substantially within the range of (approximately) 50 cm to (approximately) 170 cm. Overall lengths outside this range, however, would not be beyond the scope of the present disclosure. It is also envisioned that kinking, binding, and other such undesirable deformation may be inhibited (if not entirely prevented) by utilizing one or more flexible and/or resilient materials in construction of the catheter C.

It is envisioned that the pull wire(s) 382 may be secured (connected) to the catheter C in any suitable manner. For example, it is envisioned that the pull wire(s) 382 may be secured to an outer surface 384 of the catheter C or that the pull wire(s) 382 may extend through the delivery catheter 100 (e.g., within a corresponding (helical or partially helical) channel 386 formed in a wall 388 (outer wall) of the catheter C such that the pull wire(s) 382 are embedded within the catheter C).

Upon the application of a (pulling) force to the pull wire(s) 382, the catheter C experiences angular (torsional) deflection (displacement), whereby the catheter C rotates about the longitudinal axis X. The rotation facilitated by the torsional force applied to the catheter C via the pull wire(s) 382 allows for precise control over the rotational positions of the catheter C (e.g., the side hole(s) 370) and, thus, the supplemental medical (therapeutic) device(s) inserted therethrough (FIG. 29) to not only facilitate alignment of the side hole(s) 370 with the vascular abnormality A, but facilitate insertion alignment of the supplemental medical (therapeutic) device(s) and/or the embolic device 380 into the vascular abnormality A.

To facilitate the application of force to the pull wire(s) 382, in certain embodiments, the catheter C may include (or may be connected to) one or more corresponding activating mechanisms 390 (e.g., such that the number of pull wires 382 corresponds to the number of activating mechanisms 390). The activating mechanism(s) 390 are connected to the pull wire(s) 382 and may include any structure or mechanism suitable for the intended purpose of applying torsional force thereto sufficient to cause rotation of the catheter C as necessary or desired, such as, for example, rotating wheels, pulley systems, ratchet mechanism, levers, or the like. In certain embodiments, it is envisioned that the activating mechanism(s) 390 and/or the pull wire(s) 382 may include one or more stop locks (or other such structures) to maintain the rotational position(s) of the pull wire(s) 382 and the catheter C.

In certain embodiments of the disclosure, it is also envisioned that the activating mechanism(s) 390 may be omitted, and that force may be manually applied to the pull wire(s) 382 to facilitate rotation of the catheter C.

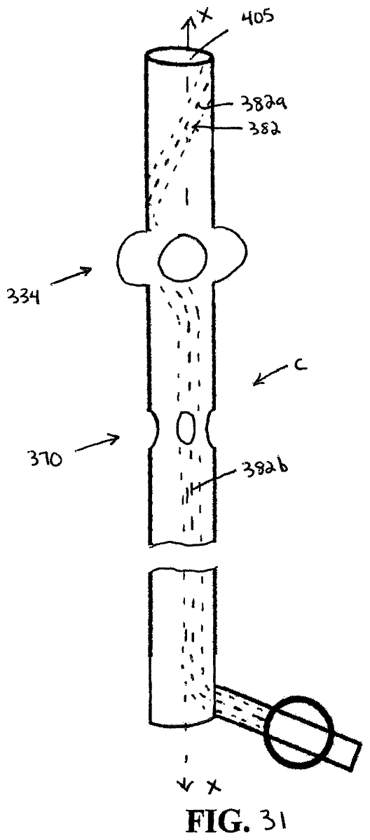

In the embodiment illustrated in FIG. 30, the catheter C includes a single pull wire 382 and a single activating mechanism 390, which allows for rotation of the catheter C in a single direction only (e.g., counterclockwise in the direction indicated by arrow 1). In alternate embodiments of the disclosure, however, it is envisioned that the catheter C may include a plurality of pull wires 382 and a plurality of corresponding activating mechanisms 390 to facilitate rotation of the catheter C in a plurality of directions (e.g., clockwise and counterclockwise). More specifically, FIG. 32 illustrates an embodiment of the disclosure in which the catheter C includes a (first) pull wire 382i that is connected to a (first) activating mechanisms 390i to thereby apply a (first) torsional force to the catheter C and rotate the catheter C in a (first) direction indicated by the arrow 1 and a (second) pull wire 382ii that is connected to a (second) activating mechanisms 390ii to thereby apply a (second) torsional force to the catheter C and rotate the catheter C in a (second) direction indicated by the arrow 2. In other embodiments, the two or more pull (or push) wires can apply a torsional force to rotate the catheter in the same direction.

It is envisioned that the pull wires 382i, 382ii may be connected to the catheter C in any suitable locations. For example, to facilitate rotation in (generally) opposing directions 1, 2, it is envisioned that the pull wires 382i, 382ii may be connected to the catheter C at respective connection points 392i, 392ii that are positioned in (generally) diametric opposition.

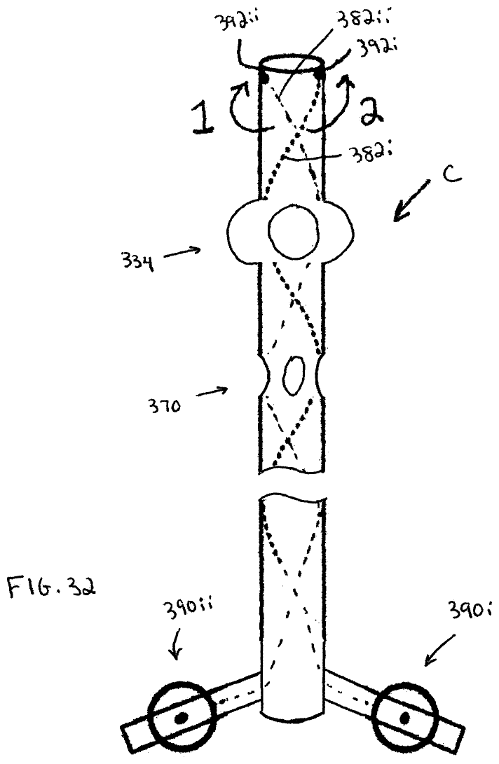

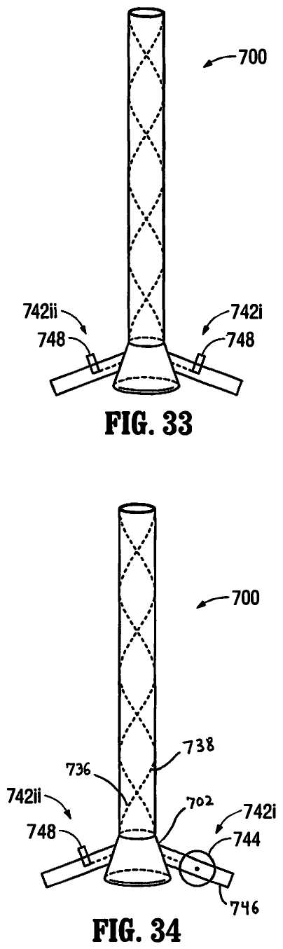

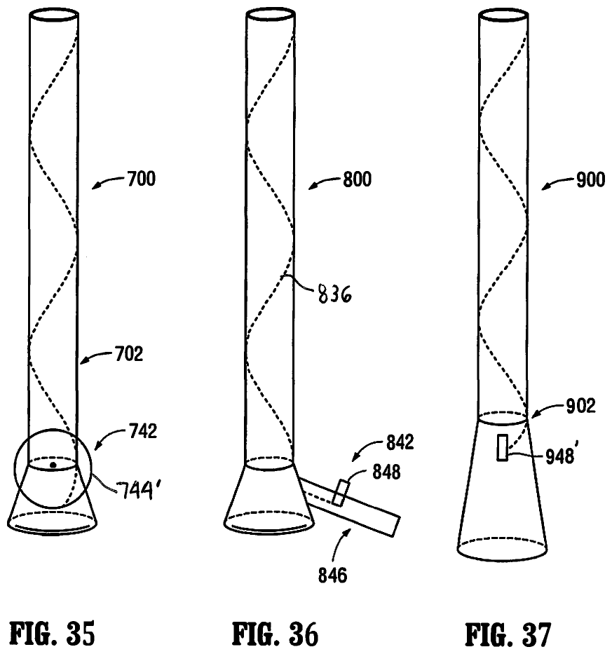

In the particular embodiment illustrated in FIG. 34, the catheter 700 includes an activating mechanism 74.2i that is configured as a wheel 744 such that force is applied to the pull wire 736 via rotation of the wheel 744. An activating mechanism for pull wire 738 can also be in the form of a wheel or in the form of a lever (lever 748 of activating mechanism 742ii). Alternatively, a lever 748 can be provided for each pull wire as shown in FIG. 33. The activating mechanism 742 is supported on an access port (branch) 746 that extends laterally outward from the body 702 of the catheter 700 (e.g., to support in the insertion of one or more medical devices, guide wires, etc.). It should be appreciated, however, that the activating mechanism 742 may be positioned in any suitable location. For example, FIG. 35 illustrates an alternate embodiment that is devoid of the access port 746 in which the activating mechanism (e.g., wheel 744′) is supported directly on the body 702 of the delivery catheter 700.

FIG. 36 illustrates another embodiment of the disclosure in which a single pull wire is utilized. The activating mechanism 842 of catheter 800 is configured as a movable (e.g., pivotable) lever 848 such that force is applied to the pull wire 836 via deflection of the lever 848 (e.g., pivotable and/or axial movement of the lever). Although shown as being supported on the access port 846 in FIG. 31, it should be appreciated that the lever 848 may be positioned in any suitable location. For example, FIG. 37 illustrates another embodiment that is devoid of the access port 846 in which the lever 948′ is supported directly on the body 902 of the catheter 900.

Further details of the torsional wires are disclosed in application Ser. No. 17/214,021, filed Mar. 26, 2021, the entire contents of which are incorporated herein by reference.

Although pull wires are disclosed herein for the steerable bendable segment(s) and for changing the rotational position of the catheter (torsional wires), it is also contemplated that push wires could alternatively be provided such that pushing of the wire bends the steerable segment and/or pushing of the wire changes the rotational position of the catheter. The various medical devices (e.g., catheters, stents, hypotubes, guide wires, etc.) and procedures described herein may be utilized (combined) with the multiple circumferential balloon catheter previously described by Walzman (US 2020/10,543,015) to facilitate additional precision when orientating a delivery catheter in a desired (rotational) orientation within a blood vessel (e.g., at or adjacent to an aneurysm or the neck of an aneurysm).

It is envisioned that the various devices described herein may (optionally) include one or more steerable segments that are deflectable via one or more pull wires that extend fully or partially embedded within the wall of the device to (facilitate insertion, removal, and/or increased precision in the placement of the device). While the following discussion is provided in the context of the catheter 1100, it should be appreciated that the principles, elements, and structures described herein below may be incorporated into any of the devices described herein (e.g., the hypotube, the primary delivery catheter, the delivery device, the balloon catheter, the secondary delivery device the secondary delivery catheter, etc.).

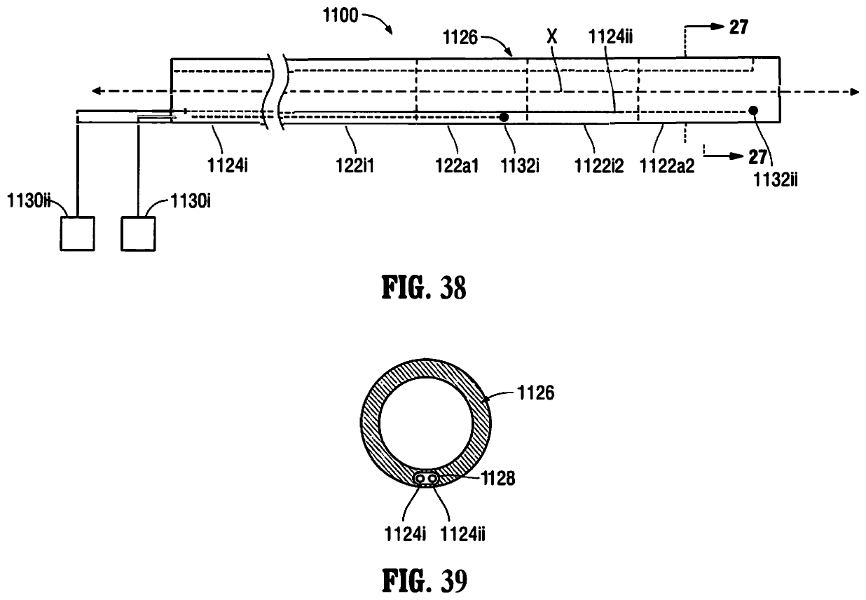

With reference now to FIGS. 38-40, in the illustrated embodiment, the delivery catheter 1100 includes a plurality of segments 1122 and a plurality of (first) pull wires 1124. More specifically, the catheter 1100 includes a plurality of inactive (passive) segments 1122i and a plurality of active (steerable, deflectable, articulable) segments 1122a that are connected to the plurality of pull wires 1124 and spaced along the longitudinal axis X of the catheter 1100. The inactive segments 1122i and the active segments 1122a are arranged in a staggered pattern such the delivery catheter 1100 alternates between inactive segments 1122i and active segments 1122a.

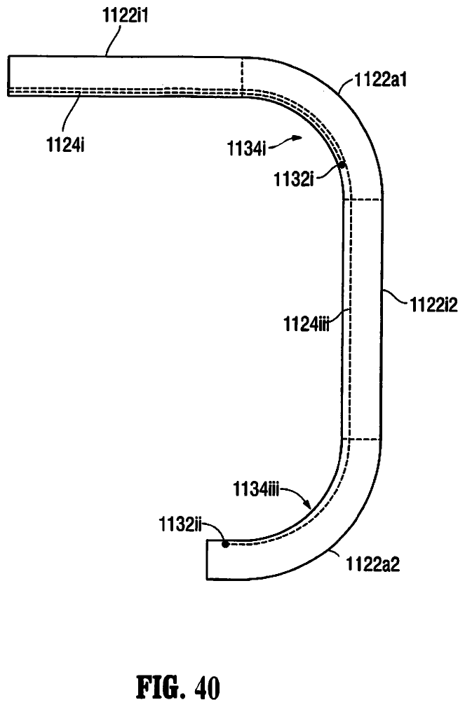

In the particular embodiment shown, each active segment 1122a is connected to a corresponding (single) pull wire 1124 that extends through (e.g., within) the body 1102 of the catheter 1100 (e.g., within an outer wall 1126 thereof) such that pull wires 1124 correspond in number to the active segments 1122a and extend in (generally) parallel relation to the longitudinal axis X of the catheter 1100. Upon the application of an axial (pulling) force to each of the pull wires 1124, the corresponding active segment 1122a is deflected (articulated) to thereby reconfigure (actively steer) the catheter 1100 between a first (initial, normal) configuration (FIG. 38), in which the catheter 1100 includes a (generally) linear configuration, and a second (subsequent, deflected) configuration (FIG. 40), in which the catheter 1100 includes a non-linear configuration.

The use of a single pull wire 1124 in connection with each active segment 1122a reduces the requisite number of pull wires 1124, thus reducing complexity in both construction and operation of the catheter 1100. It is also envisioned that multiple, independently movable pull wires 1124 may be included in other embodiments. In the particular embodiment illustrated, each pull wire 1124 is received within a corresponding channel 1128 (FIG. 39) that extends through the outer wall 1126 in (generally) parallel relation to the longitudinal axis X (e.g., such that the pull wires 1124 are embedded within the delivery catheter 100).

To facilitate the application of axial force to the pull wires 1124, in certain embodiments, the delivery catheter 1100 may include (or may be connected to) a plurality of corresponding (first) activating mechanisms 1130 (e.g., such that the number of pull wires 1124 corresponds to the number of activating mechanisms 1130). In the particular embodiment illustrated, the catheter 1100 includes a (first) activating mechanism 1130i that is connected to the pull wire 1124i and a (second) activating mechanism 1130ii that is connected to the pull wire 1124ii. The activating mechanisms 1130 may include any structure or mechanism suitable for the intended purpose of applying the axial force to the pull wires 1124 required to deflect the catheter 1100 as necessary or desired, such as, for example, rotating wheels, pulley systems, ratchet mechanism, levers, or the like. In certain embodiments, it is envisioned that the activating mechanism(s) 1130 and/or the pull wires 1124 may include one or more stop locks (or other such structures) to maintain the position(s) of the pull wires 1124 and the corresponding segments 1122.

In certain embodiments of the disclosure, it is also envisioned that the activating mechanism(s) 1130 may be omitted and that force may be manually applied to the pull wires 1124 to facilitate articulation of the delivery catheter 1100.

In certain embodiments, it is envisioned that the active segments 1122a, the pull wires 1124, and the activating mechanisms 1130 may be configured (and connected) such that each pull wire 1124 may be individually acted upon to deflect (steer) the corresponding segment 1122a in a single direction only. In other embodiments, it is envisioned that pull wires 1124 may be provided on various circumferential surfaces of the delivery catheter 1100 to facilitate steering in various directions.

In the particular embodiment illustrated, the catheter 1100 includes a first inactive segment 1122il; a first active segment 1122al that is located distally of the segment 1122il; a second inactive segment 12212 that is located distally of the segment 1122al; and a second active segment 1122a2 that is located distally of the segment 112212. Additionally, the catheter 1100 includes respective first and second pull wires 1124i, 1124ii that are located within the channel 1128 (FIG. 39). It is also envisioned, however, that the first and second pull wires 1124i, 1124ii may be located within separate channels 1128 (e.g., such that the number of channels 1128 corresponds to the number of pull wires 1124).

The pull wires 1124i, 1124ii are connected to the segments 1122al, 1122a2 at connection points 1132i, 1 132ii (in addition to the activating mechanism 1130i, 1130ii), respectively, so as to facilitate reconfiguration of the catheter 1100 between the first configuration (FIG. 38) and the second configuration (FIG. 40). More specifically, upon reconfiguration of the delivery catheter 1100, the active segments 1122ai, 122aii define respective first and second bends 1134i, 1134ii (FIG. 40), which may be either substantially similar (e.g., identical) or dissimilar depending, for example, upon the particular configuration of the segments 1122al, 1122a2, the materials of construction used in the delivery catheter 1100, the particular requirements of the delivery catheter 1100 dictated by the endovascular procedure, etc. Although the bends 1134i, 1134ii are each illustrated as being (approximately) equal to 90 degrees in FIG. 40, depending upon the particular configuration of the segments 1122al, 1122a2, the requirements of the endovascular procedure, the particular anatomy of the patient's vasculature, etc., it is envisioned that the bends 1134i, 1134ii may lie substantially within the range of approximately 0 degrees to approximately 270 degrees. For example, in one particular embodiment, it is envisioned that the segment 1122al may be configured such that the bend 1134i lies substantially within the range of approximately 0 degrees to approximately 180 degrees (e.g., approximately 90 degrees to approximately 180 degrees) and that the segment 1122a2 may be configured such that the bend 1134ii lies substantially within the range of approximately 0 degrees to approximately 270 degrees (e.g., approximately 90 degrees to approximately 270 degrees).

In the particular embodiment illustrated, the connection points 1132i, 1132ii are shown as being in (general) angular alignment (e.g., along a circumference of the catheter 100), which facilitates deflection of the segments 1122al, 1122a2 in similar (e.g., identical) directions, as seen in FIG. 40. It is also envisioned, however, that the connection points 1132i, 1132ii may be angularly offset so as to facilitate deflection of the segments 1122al, 1122a2 in dissimilar directions. For example, the connection points 1132i, 1132ii may be oriented in (generally) diametric opposition such that the bends 1134i, 1134ii respectively defined by the segments 1122al, 1122a2 curve in (generally) opposite directions.

It is also envisioned that the delivery catheter 100 may include one or more (second) pull wires that are connected (secured, anchored) to the catheter, which may either supplement or replace the pull wire(s) 124 (FIGS. 38-40). The second pull wire(s) facilitate the selective application of a torsional (twisting) force to the delivery catheter and, thus, rotational deflection of the delivery catheter along all or a portion of the length thereof (e.g., at or adjacent to the distal end hole) to vary the angular position of the catheter as described above.

It is envisioned that the pull wires for rotation disclosed herein can fully or partially extend about the longitudinal axis X, i.e., extend 360 degrees, less than 360 degrees or greater than 360 degrees (more than one spiral).

In some embodiments, the pull wires can be straight in part or most of the device and spiral or substantially spiral (wound substantially helically) in only part of its length. In some embodiments, the substantially spiraling (substantially helical winding) portion of the pull wires extends until at or near the distal end of the device. The substantially spiraling (substantially helical winding) of the wires can be configured so rotation occurs in a desired segment of the device, for example, to rotate the orientation of the distal end hole as desired. In accordance with one method of the present invention, a catheter with at least two independent steerable zones (bendable segments), with each steer zone controlled by at least one wire positioned substantially in the wall of the catheter is utilized. In a preferred embodiment, both zones steer to bend the catheter in the same direction. The method includes a) introducing the primary catheter via a percutaneous technique, over a wire, in a right radial artery; b) then advancing the primary catheter over a wire separately into the right subclavian artery; c) from where, advancing an inner catheter into the right vertebral artery, into the innominate artery, and from where, the wire can be withdrawn proximally so the distal steer zone can be used to steer the tip of the primary catheter into the right common carotid artery. The inner catheter can then be advanced as desired selectively (separately) into the right internal carotid artery and/or right external carotid artery. Next, the inner catheter and wire are withdrawn, unbending the primary catheter, and the primary catheter is advanced over a wire (or without a wire) into the aortic arch. The wire (if used) is next withdrawn from the inner primary catheter and repositioning via steering of a distal steer zone so the distal end is engaged in the proximal left common carotid artery. An inner catheter can then be advanced as desired selectively (separately) into the left internal carotid artery and/or left external carotid artery. If a wire is used, alternatively or additionally the inner catheter and wire are withdrawn, unbending the primary catheter, and then advancing the primary catheter over a wire (or without a wire) into the aortic arch, withdrawing the wire (if used) from the primary catheter and repositioning via steering of a distal steer zone so the distal end is engaged in the proximal left subclavian artery. The inner catheter can then be advanced as desired selectively (separately) into the left vertebral artery.

In these methods, the torsional wire(s) e.g., spiral wire(s), (if provided) attached at a distal portion of the catheter can be pulled or pushed to rotate a distal segment of the catheter. In addition or in lieu of the torsional wire(s), a wire(s) attached to bending/steering segments of the catheter can be pushed or pulled to bend/steer a segment of the catheter.

In these methods, the delivery catheter can have one or more side holes in a catheter segment that rotates to place the side hole at a desired orientation relative to a lesion or a vessel to facilitate the delivery of additional catheters and/or devices through the side hole. The delivery catheter can have structure, e.g., bends, to further provide support to the additional catheters and/or devices to prevent kickback and unwanted prolapse of the catheters and/or devices during delivery.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described.

It must be noted that as used herein and in the appended claims, the singular forms “a”, “and”, and “the” include plural references unless the context clearly dictates otherwise.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

Additionally, persons skilled in the art will understand that the elements and features shown or described in connection with one embodiment may be combined with those of another embodiment without departing from the scope of the present disclosure and will appreciate further features and advantages of the presently disclosed subject matter based on the description provided.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1. A catheter configured for use during an endovascular procedure to treat a vascular abnormality in a blood vessel, the catheter comprising:
a lumen configured to receive a supplemental medical device to facilitate treatment of the vascular abnormality;
at least one side hole extending through the wall of the catheter and in communication with the lumen, the at least one side hole configured to receive the supplemental medical device such that the supplemental medical device is extendable into the blood vessel through the at least one side hole to treat the vascular abnormality; and
a first wire for applying a torsional force to the catheter and thereby rotate at least a segment of the catheter to vary a rotational position of the at least one side hole.

2. The catheter of claim 1, further comprising a second wire for deflecting a segment of the catheter for steering the catheter.

3. The catheter of claim 1, wherein the at least one side hole includes a plurality of side holes spaced apart along a circumference of the catheter.

4. The catheter of claim 1, further comprising at least one balloon element supported on an outer surface of the catheter and an inflation lumen extending through a wall of the catheter to selectively inflate the at least one balloon element.

5. The catheter of claim 1, wherein the first wire is embedded in a wall of the catheter.

6. The catheter of claim 2, wherein the second wire is embedded in a wall of the catheter.

7. The catheter of claim 4, wherein the at least one balloon element includes a plurality of balloon elements spaced apart along a circumference of the catheter.

8. (canceled)

9. The catheter of claim 1, further comprising an additional wire for applying a torsional force to the catheter and thereby rotate the catheter to vary a rotational position of the at least one side hole.

10. (canceled)

11. The catheter of claim 9, wherein one or both of at least a segment of the first wire and at least a segment of the additional wire are in a substantially spiral configuration.

12. A catheter configured to receive a supplemental medical device to facilitate treatment of a vascular narrowing within a blood vessel, the catheter comprising:
a proximal hole;
a distal hole;
a lumen extending between the proximal hole and the distal hole and configured to receive the supplemental medical device; and
at least one side hole in communication with the lumen, wherein the at least one side hole is configured to receive the supplemental medical device such that the supplemental medical device is extendable into the blood vessel through an elected side hole to increase access to a target site in the blood vessel and reduce rotational manipulation of the catheter required during an endovascular procedure;
wherein the catheter provides support to the supplemental medical device to prevent kickback and prolapse.

13. (canceled)

14. The catheter of claim 12, further comprising at least one wire configured to apply torsional force to the catheter and thereby rotate the catheter to vary a rotational position of the at least one side hole.

15. The catheter of claim 9, wherein the first wire is configured to rotate the catheter in a first direction and the additional wire is configured to rotate the catheter in a second direction generally opposite to the first direction.

16. The catheter of claim 9, wherein the first wire and the additional wire are configured to rotate the catheter in a first direction.

17. The catheter of claim 12, wherein the catheter includes at least one steerable bending segment.

18-19. (canceled)

20. The catheter of claim 1, wherein the at least one side hole includes a plurality of side holes staggered along a longitudinal axis of the catheter such that the plurality of side holes are spaced circumferentially and axially from each other in a generally helical arrangement.

21. The catheter of claim 12, further comprising at least one balloon element positioned proximally of the distal hole.

22. The catheter of claim 21, wherein the at least one balloon element comprises a plurality of balloon elements arranged along a circumference of the catheter such that the plurality of balloon elements are spaced circumferentially from each other.

23. The catheter of claim 21, wherein the at least one balloon element comprises a plurality of balloon elements circumferentially aligned such that the plurality of balloon elements are arranged in a band.

24. The catheter of claim 21, wherein the at least one balloon element comprises a plurality of balloon elements staggered along a longitudinal axis of the catheter such that the plurality of balloon elements are spaced circumferentially and axially from each other in a generally helical arrangement.

25. The catheter of claim 12, in combination with an outer catheter having a plurality of balloon elements arranged along a circumference of the outer catheter.

26-41. (canceled)
